(19) United States
(12) Patent Application Publication
(10) Pub. No.: US 2010/0267822 A1
(43) Pub. Date: Oct. 21, 2010

(54) AURONES AS ESTROGEN RECEPTOR MODULATORS AND THEIR USE IN SEX HORMONE DEPENDENT DISEASES

(76) Inventors: Annie George D/O V.K. George, Selangor (MY); Bärbel Köpcke, Dortmund (DE); Ernst Roemer, Bucha (DE); Jens Bitzer, Dortmund (DE); Joachim Hans, Dortmund (DE); Joerg Gruenwald, Berlin (DE); Matthias Gehling, Leichlingen (DE); Philipp Wabnitz, Dusseldorf (DE); Tengku Shahrir bin Tengku Adnan, Kuala Lumpur (MY); Torsten Grothe, Bochum (DE)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 12/749,024

(22) Filed: Mar. 29, 2010

Related U.S. Application Data
(60) Provisional application No. 61/164,055, filed on Mar. 27, 2009.

(51) Int. Cl. A61K 31/343; C07D 307/83; A61P 25/00; A61P 25/24; A61P 25/28; A61P 27/02; A61P 9/12; A61P 19/08; A61P 35/00; A61P 9/00; A61P 15/00; A61P 5/30

(52) U.S. Cl. 514/470; 549/466

(57) ABSTRACT

The invention relates to aurones and extracts comprising them useful in the prophylactic and/or therapeutic treatment of an animal (including a human) with an estrogen receptor (ER) related disease or condition of the animal or human body, as well as methods, uses and other inventions related thereto.

### RELATED APPLICATIONS

The present application claims the benefit of U.S. Provisional Application No. 61/164,055 filed Mar. 27, 2009, which is incorporated herein in its entirety by reference.

### FIELD OF THE INVENTION

The invention relates to aurones and extracts comprising them useful in the prophylactic and/or (especially) therapeutic treatment of an animal (including a human) with an estrogen receptor (ER) related disease or condition of the animal or human body, as well as methods, uses and other inventions related thereto as described below and in the claims

### BACKGROUND OF THE INVENTION

The estrogen receptor (ER) is a member of the nuclear hormone receptor superfamily. Phylogenetic analysis has identified several subfamilies within this superfamily: type I (“classical” or “steroid”) receptors include those for progestins (PR), estrogens (ER), androgens (AR), glucocorticoids (GR), and mineralocorticoids (MR), whereas type II receptors encompass those for thyroid hormone (TR), all-trans retinoic acid (RAR), 9-cis retinoic acid (RXR), and vitamin D3 (VDR) (N. McKenna et al, Endocrine Rev. 1999; 20(3): 321-344).

Until recently, it was assumed that estrogen binds to a single estrogen receptor (ER) in cells. However, in the end of the last century, the discovery of a second type of ER in rat (G. Kuiper et al., Proc Natl Acad Sci USA 1996; 93: 5925-30), mouse (G. Tremblay et al. Endocrinology 1997; 11: 353-65), and human (S. Mosselman et al., FEBS Lett 1996; 392: 49-53) is reported. This new receptor was termed ERbeta, resulting in the classical ER being referred to as ERalpha. The two receptors are not isoforms of each other (J. Couse & S. Kenneth, Endocrine Rev 1999; 20(3): 358-417).

Other low affinity binding sites, so called nuclear type II estrogen bindings sites, (EBS) were found (Markaverich et al., Cancer Res 1984; 44: 1515-1519; J Steroid Biochem 1988; 30: 71-8; WO 1991/017749). However, despite the abundant presence of EBS in numerous tissues and tumors, the protein was very difficult to isolate. The EBS were thought to be associated with tyrosinase-like activity (Adlercreutz, Crit. Rev Clinical Lab Sci 2007; 44(5-6): 483-525 and further references), but they were later separated from this (Densmore et al., Steroids 1994:59:282-7). EBS have also been found in MCF-7 human breast cancer cells. Recently they were identified as histone H4 (Shoulars et al., Biochem Biophys Res Commun 2002; 296: 1083-90). The same authors explicitly explain the difference between ERalpha, ERbeta on one side and EBS on the other one. It is known that some ligands for EBS, such as bioflavanoids, have no affinity for ERalpha or ERbeta (Raghvendra et al., Am J Physiol Renal Physiol 2001; 280: F365-88).

Estradiol (E2) is known to bind to both ERalpha and ERbeta with equal affinity. There are however ligands (such as e.g. tamoxifen) that exhibit differential affinities towards the two receptor types (Riggs and Hartmann, N Engl J Med. 2003 348(7):618-29). Additionally, these ligands may act either as agonists or antagonists of receptor activity, depending on the specific tissue or cell type, and the existence and relative concentration of co-agonists.

This complex system of estrogen receptor activity modulation has given rise to the development of an entirely new class of drugs, referred to as Selective Estrogen Receptor Modulators or SERMs, which are therapeutically useful agents to treat or prevent: osteoporosis, hypocalcaemia of malignancy, bone loss or bone fractures (V. Jordan et al., Natl Cancer Inst 2001; 93(19):1449-57; N. Bjarnason et al., Osteoporosis Int 2001; 12(11):922-3; S. Fentiman, Eur J Cancer 1992; 28:684-685; G. Rodan et al., Science 2000; 289: 1508-14); periodontal disease or tooth loss as well as Paget's disease (G. Rodan et al., Science 2000; 289: 1508-14); cartilage degeneration, rheumatoid arthritis or osteoarthritis (A. Badger et al., J Pharmacol Exp Ther. 1999; 291(3): 1380-6); cardiovascular disease, restenosis, lowering levels of LDL cholesterol and inhibiting vascular smooth muscle cell proliferation (M. Nuttall et al., Endocrinology 1998; 139(12):5224-34; V. Jordan et al., Natl Cancer Inst 2001; 93(19):1449-57; J. Guzzo, Clin Cardiol 2000; 23(1):15-7; T. Simoncini & A. Genazzani, Curr Opin Obstet Gynecol 2000; 12(3):181-7); ischemic damage (Carswell et al., Am J Physiol Heart Circ Physiol 2004; 287(4): H1501-4; obesity (F. Picard et al., Int J Obes Relat Metab Disord. 2000; 24(7):830-40).

Estrogen receptor beta has been reported to have a role in the regulation of vascular function and blood pressure, see Zhu et al., Science 2002: 1295(5554): 505-508.

The contribution of estrogen receptors in the modulation of emotional processes, such as anxiety, has been described in the art, see W. Krezel et al., Proc Natl Acad Sci USA 2001; 98 (21):12278-82.

The use of estrogen receptor modulators to treat sexual dysfunction has been described in the art, see E. Baulieu et al., PNAS 2000, 97(8): 4279-4282; R. Spark, Fertility & Sterility, 2002; 77(4): 19-25.

In models, estrogen has been shown to have beneficial effects on cognitive function, such as relieving anxiety and depression and treating or preventing Alzheimer's disease. Estrogen affects the central nervous system by increasing cholinergic functioning, neurotrophin and neurotrophin receptor expression. Estrogen also increases glutamergic synaptic transmission, and provides neuroprotection. Thus, the estrogen receptor modulators of the present invention could be beneficial for improving cognitive functioning or treating age-related mild cognitive impairment, attention deficit disorder, sleep disorders, irritability, impulsivity, anger management, multiple sclerosis and Parkinson's disease (H. Sawada & S. Shimohama Endocrine. 2003; 21(1):77-9; L. McCullough & P. Rum, Trends Endocrinol Metab. 2003; 14(5):228-35).

The utility of SERMs to prevent the impairment of cognitive functioning is known in the art (K. Yaffe et al., N. Eng. J. Med. 2001; 344: 1207-1213).

The utility of estrogens to prevent depression has been described by S. Carranza-Liram & M. Valentino-Figueroa, Int J Gynnaecol Obstet 1999; 65(1):35-8. Specifically, estrogen receptor beta (ERβ) selective agonists would be useful in the treatment of anxiety or depressive illness, including depression, perimenopausal depression, post-partum depression, premenstrual syndrome, manic depression, anxiety, dementia, and obsessive compulsive behavior, as either a single agent or in combination with other agents. Clinical studies have demonstrated the efficacy of the natural estrogen, 17β-estradiol, for the treatment of various forms of depressive illness; see P. Schmidt et al., Am J Obstet Gynecol 2000; 183:414-25; C. Soares et al., Arch Gen Psychiatry, 2001; 58:537-8; which are hereby incorporated by reference. C. Bethea et al., Endocrine 1999; 11:257-67, which is hereby incorporated by reference, have suggested that the anti-depressant activity of estrogen may be mediated via regulation of serotonin synthesis in the serotonin containing cells concentrated in the dorsal raphe nucleus.

There remains a need for safe and effective compositions for the treatment of ER related diseases in subjects such as humans. The problem to be solved by the present invention is therefore to find novel compositions or compounds useful in the treatment of ER related diseases.

Smilax is a genus of about 600 species of climbing flowering plants, many of which are woody and/or thorny, in the monocotyledon family Smilacaceae, native throughout the tropical and warm temperate regions of the world. On their own, Smilax plants will grow as a shrub, forming dense impenetrable thickets. They will also grow over trees and other plants up to 10 m high using its hooked thorns to hang on to and scramble over branches. The leaves are heart shaped and vary from 4-30 cm long in different species.

Extracts (predominantly from the roots) of Smilax species have been used to treat various conditions. Therapeutic properties like anti-inflammatory, antifungal, antipruritic, anti-rheumatic, antiseptic, aphrodisiac, wound healing, stimulant, diuretic, diaphoretic, depurative, sudorific, tonic are attributed to them. Traditional/Ethnobotanical use is described for more then 40 Smilax species (http://www.ars-grin.gov/duke/). For selected Smilax species this is:

Smilax aristolochiaefolia (Cancer, Depurative, Dyspepsia, Eczema, Fever, Gonorrhea, Kidney, Leprosy, Rash, Rheumatism, Scrofula, Skin, Sudorific, Syphilis), Smilax aristolochiifolia (Depurative, Diaphoretic, Syphilis, Tonic, Wound), Smilax china L. (Aphrodisiac, Dermatosis, Gonorrhea, Parturition, Rheumatism, Syphilis, Tonic), Smilax china (Abscess, Alexiteric, Antidote, Aphrodisiac, Arthritis, Asthma, Boil, Cancer, Carminative, Cold, Debility, Demulcent, Depurative, Diaphoretic, Diarrhea, Diuretic, Enteritis, Flux, Gout, Gravel, Malaria, Menorrhagia, Refrigerant, Rheumatism, Skin, Stimulant, Sudorific, Syphilis, Tonic, Urogenital, Venereal) Smilax glabra (Abscess, Antidote, Arthritis, Boil, Cystitis, Dysentery, Furuncle, Lymphadenopathy, Rheumatism, Skin, Sore, Syphilis, Venereal), Smilax medica (Antidote, Malignancy, Rheumatism, Scrofula, Skin, Stimulant, Sudorific, Venereal), Smilax ornata (Rheumatism, Scrofula, Skin, Tonic), Smilax scobinicaulis (Arthritis, Rheumatism, Skin, Sore), Smilax sieboldii (Arthritis, Rheumatism, Skin, Sore), Smilax zeylanica (Abscess, Ache (Bones), Anodyne, Cachexia, Cholera, Dysentery, Dysuria, Fever, Gravel, Measles, Ophthalmia, Skin, Smallpox, Sore, Swelling, Syphilis, Venereal)

Smilax myosotiflora is a very damage tolerant thorny plant capable of growing back from its rhizomes after being cut down or burned down by fire. It grows wild in the tropical forest in South East Asia, namely but not limited to Malaysia, Indonesia and Southern Thailand.

In Malaysia, the tuber or rhizome is used as an aphrodisiac and sexual tonic and to treat fevers. It is claimed that it increases the production of testosterone in elderly men, hence, improving sperm production and its viscosity, vitality and sexual strength. It also restores vitality and libido in women, firming the vagina especially after delivery and arresting vaginal discharge. The leaves and fruits are used to treat syphilis.

In traditional preparation, the rhizome is boiled by itself or mixed with Tongkat Ali root, horny goat weed (Epimedium) or Kacip Fatimah, Manjakani, Serapat, and other herbs to enhance the efficacy. The tonic is taken regularly once or twice a day. In modern preparation, phyto-chemicals from the Ubi Jaga rhizome are extracted, frozen or spray-dried. The extracts are similarly mixed with other herb-extracts and formulated separately for men or women.

The leaves, fruits and rhizomes of Smilax myosotiflora were used to treat syphilis i.e. a bacterial infection. The rhizome is ingested as an aphrodisiac. The leaves and fruits of are used to relieve fever (http://khenerg.com/faq.html).

There are several registered products on the market containing Smilax myosotiflora (Ubi jaga) in mixtures with other medicinal plants (Malayan Ministery of Health; http://search.moh.gov.my). Mixtures with e.g. Tongkat Ali (Eurycoma longifolia) are widely promoted on the Internet as to be used as an aphrodisiac. The main use is to increase male sexual power, increase general health and energy, and secondarily to improve nerve system and blood circulation.

Aurones are natural molecules which belong to the family of flavonoids, and which are structurally isomers of flavones (Boumendjel, Current Med. Chem. 2003). Systematically they were named as benzylidenebenzofuran-3(2H)-ones.

Aurones are broadly widespread in the plant kingdom, particularly in fruits and flowers in which they contribute to their coloration. Table 1 below contains a non-exhaustive, exemplary list of natural aurones which are found in plants. According to their substitution pattern, these aurones can be grouped into mono-, di-, tri-, tetra-, penta- and heptahydroxylated representatives which carry partially additional alkyl groups attached to core. The hydroxyl groups are free, methylated or carry sugar moieties.

Table 1 shows some aurone type compounds and their sources. (Dictionary of Natural Products, Chapman & Hall, 2008)

TABLE 1

| structure-type | quantity of entries | sources |
|---|---|---|
| 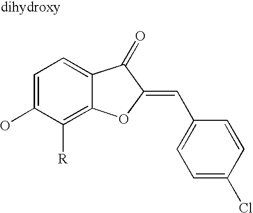 | 4; R = H | Glycine max (soybean) and Lygos raetam |
| | 2; R = methyl | Pterocarpus marsupium |

trihydroxy

| | | |
|---|---|---|
| 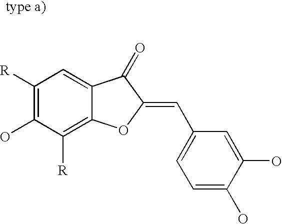 | 4; R = H 2; R = prenyl | Bidens tripartita, bidens sulphureus, Bidens laevis, Dahlia variabilis, Baeria chrysostoma, Rhus cotinus, Schinopsis, Amphipterygium adstringens, Cosmos sulphureus, Cosmos maritima, Viguiera, Zinnia, Coreopsi s, Lasthenia, Tithonia, Butea frondosa, Dipteryx odorata, Broussonetia papyrifera |
| 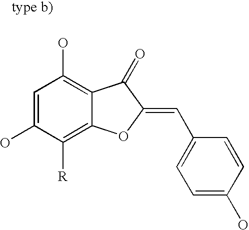 | 4; R = H | Asparagus gonocladus, Limonium sp., Pterocarpus marsupium, Pterocarpus santalinus, Asarum longerhizomatosum |
| | 2; R = methyl | Pterocarpus marsupium |
| 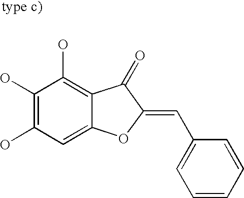 | 2 | Cephalocereus senilis |

tetrahydroxy

| | | |
|---|---|---|
| 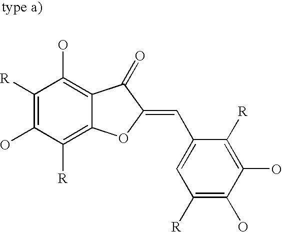 | 9; R = H | Oxalis cernua, Chirita micromusa, Limonium bonduellii, Petrocosmea kerrii, Mussaenda hirsutissima, Antirrhinum majus, Antirrhinum nuttalianum, Linaria maroccona, Marchantia berteroana, Marchantia polymorpha, Conocephalum supradecompositum, Carrpos sphaero- carpus, Mussaenda hirsutissima, Pterocarpus marsupium, Melanorrhoea spp., Cyperus capitatus |
| | 6; R = methyl or | Antiaris toxicaria, Cyperus capitatus |
| | prenyl | |
| 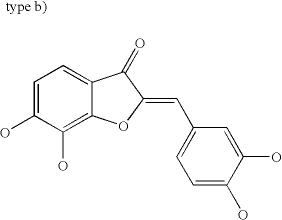 | 13 | Coreopsis maritima, Coreopsis gigantea, Coreopsis tinctoria, Baeria chrysostoma, Zinnia linearis, Bidens bipinnata, Bidens pilosa, Microglossa pyrifolia, Coreopsis grandiflora, Vaccinium oxycoccus, Cyperus scariosus |
| 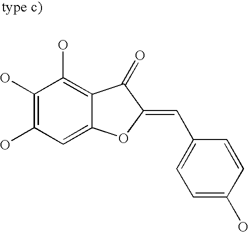 | 2 | Helianthus annuus |
| 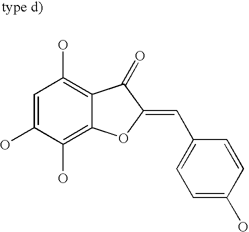 | 1 | Picris echoides |
| 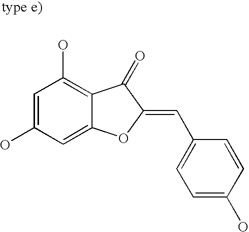 | 1 | Diospyros melanoxylon |

pentahydroxy

| | | |
|---|---|---|
| 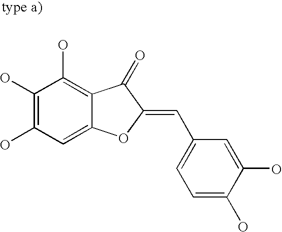 | 2 | Uvaria hamiltonii |
| 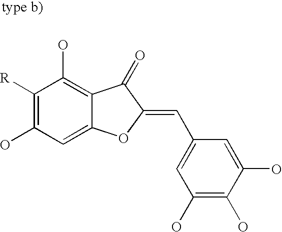 | 6; R = H | Antirrhinum nuttalianum, Linaria maroccana, Helichrysum bracteatum, Antirrhinum majus, Antirrhinum orontium, Linaria sp., Amomum subulatum |
| | 2; R = methyl | Pterocarpus santalinus |
| 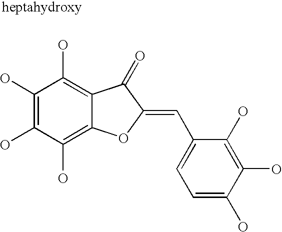 | 3 | Gomphrena agrestis |

Also known are dimers of aurones, such as disulfuretin and biaureusidin.

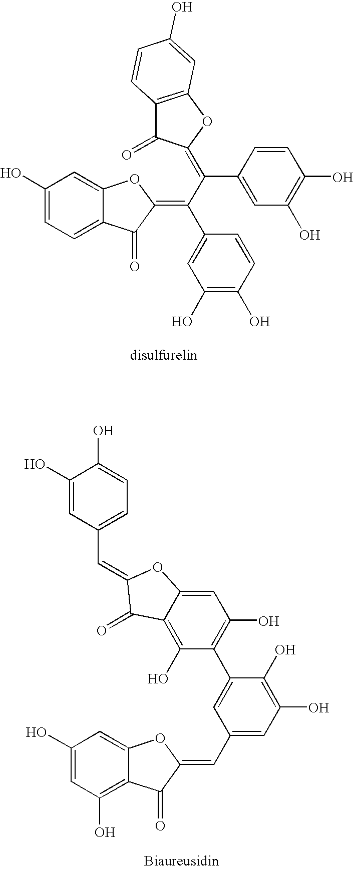

One Aurone is known to be isolated out of Smilax bracteata (Zhang 2008).

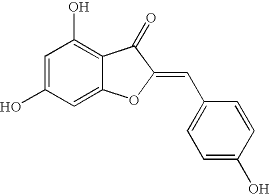

Nevertheless, till now no secondary metabolites of this class have been described with chemical structure for Smilax myosotiflora.

Aurones are known to be useful in the treatment of prostatic cancer, prostatomegaly, masculinism, breast cancer, mastopathy, uterine cancer, endometriosis, and ovarian cancer (JP H10-209268) by inhibiting 17β-hydroxysteroid-dehydrogenase activity.

2-(benzyl)-3-arylbenzofurans are known as antitumor and hypocholesterolemic agents (U.S. Pat. No. 5,354,861).

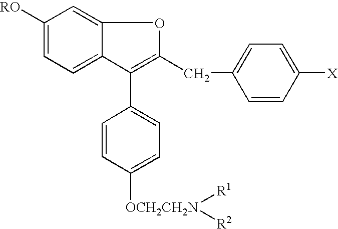

The benzofurans of the cited invention are ligands for antiestrogen-binding sites (AEBS) and display no significant interaction with the estrogen receptor (ER).

Aurones are biochemically and structurally related to flavons. The flavons are widely present in aromatic, medicinal and edible plants, and also in fruits and vegetables. In general they exist as aglycones or glycosylated at various hydroxyl groups.

In order to optimize the production of the compound of interest, aurones, liquid-liquid extraction procedure was employed. Liquid-liquid extraction, also known as solvent extraction and partitioning, is a method to separate compounds based on their relative solubilities in two different immiscible liquids, preferably only partial miscible, usually water and an organic solvent. It is an extraction of a substance from one liquid phase into another liquid phase. Liquid-liquid extraction is a basic technique in chemical laboratories, where it is preferably performed using a separatory funnel. For the enrichment of phytochemicals from a crude plant extract, usually the concentrated extract is partly dissolved in water or solvent-containing water (solvents here are co-solvents for example methanol, ethanol, propanol, isopropanol, acetone, acetonitrile or other water-miscible solvents) and extracted successively with water-immiscible solvents, preferably only partial miscible, of increasing polarity (for example, not limited to these, in the order of; 1. heptane, hexane, pentane, cyclohexane, petroleum ether; 2. diethyl ether, toluene, benzene, t-butyl methyl ether, chloroform, dichloromethane, ethyl methyl ketone, dioxane, tetrahydrofuran; 3. ethyl acetate).

Recent advances were made to evaluate the therapeutical potential of aurones in different pharmacological areas like:
Use in cancer chemotherapy (modulators of P-glycoprotein-mediated MDR; inhibition of cyclin-dependent kinases; interactions with adenosine receptors; effects through DNA scission and telomerase Inhibition)
Use in treating parasitic infections
Use in treating microbial infections
Antihormonal activity of aurones
Aurones as antidiabetics.

### DETAILED DESCRIPTION OF THE INVENTION

The present invention relates to a compound of the formula I,

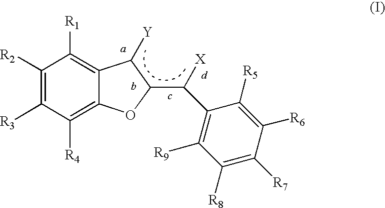

wherein each of R₁ to R₉ is, independently of the others, H, hydroxy, fluoro, chloro, bromo, iodo, C₁-C₈-alkyl, C₂-C₈-alkenyl, C₂-C₈-alkynyl, C₃-C₁₀-cycloalkyl, phenyloxy, C₁-C₈-alkoxy, C₁-C₉-alkanoyloxy, benzoyl or the radical of a C₅-C₁₂-carbohydrate bound via one of its oxygen atoms, where alkyl, alkenyl, alkynyl, cycloalkyl, phenyl, alkoxy, alkanoyloxy and benzoyl can be unsubstituted or substituted by one, two or three substituents selected independently of each other from the group consisting of —F, —Cl, —Br, —I, —OH, —OCH₃, —OCH₂CH₃, —OCOCH₃, —CH₃, —CHO, and —CO₂H, or the radical of a C₅-C₁₂-carbohydrate bound via one of its oxygen atoms, preferably with the proviso that if R₁, R₃ and R₇ each are bound via an oxygen, R₂, R₄, R₅ and R₉ each are hydrogen and one of R₆ and R₈ is bound via an oxygen, then the other of R₆ and R₈ has one of the meanings mentioned above other than H;
where one of R₁ to R₉ may, in addition, be a substitutent of the subformula IA

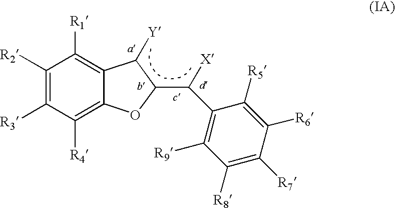

wherein one of R₁′ to R₉′ forms the bond to the rest of the molecule in formula I, while the others are, independently of each other, H, hydroxy, fluoro, chloro, bromo, iodo, C₁-C₈-alkyl, C₂-C₈-alkenyl, C₂-C₈-alkynyl, C₃-C₁₀-cycloalkyl, phenyloxy, C₁-C₈-alkoxy, C₁-C₉-alkanoyloxy, benzoyl or the radical of a C₅-C₁₂-carbohydrate bound via one of its oxygen atoms, where alkyl, alkenyl, alkynyl, cycloalkyl, phenyl, alkoxy, alkanoyloxy and benzoyl can be unsubstituted or substituted by one, two or three substituents selected independently of each other from the group consisting of —F, —Cl, —Br, —I, —OH, —OCH₃, —OCH₂CH₃, —OCOCH₃, —CHO, and —CO₂H;
or two adjacent moieties of R₁ to R₉ and of R₁′ to R₉′ together form a —O—CH₂—O— or a —O—CH₂—CH₂—O— bridge, thus forming with the two atoms to which they are bound a ring, while the other moieties are independently selected from those mentioned above;
in formula I either bond a and bond c each are a double bond, or bonds b and bond d each are a double bond, respectively;
and, if present, in subformula IA either bond a′ and bond c′ each are a double bond, or bonds b′ and bond d′ each are a double bond, respectively;
where the double bonds in formula I and, if present, subformula IA, may also be in tautomeric equilibrium (of a beta di-keto system);
X is hydrogen, oxo, hydroxy, C₁-C₃-alkoxy, especially methoxy, C₁-C₈-alkanoyloxy, especially acetyloxy, benzoyloxy or 3,4,5-trihydroxybenzoyloxy, or, if bonds a and c are double bonds in formula I and Y is oxo, can also be a moiety of the subformula IB,

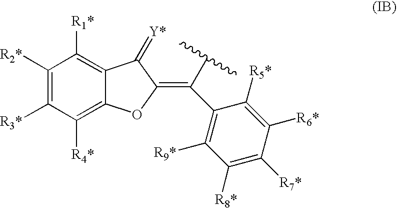

wherein the waved line indicates the end of the bond where said moiety of the subformula IB is bound to the rest of the molecule of formula I and wherein
Y* is oxo and
R₁* to R₉* are, independently of each other, H, hydroxy, fluoro, chloro, bromo, iodo, C₁-C₈-alkyl, phenyloxy, C₁-C₈-alkoxy, C₁-C₉-alkanoyloxy, benzoyl or the radical of a C₅-C₁₂-carbohydrate bound via one of its oxygen atoms;
   and Y is oxo, hydroxy or C₁-C₈-alkoxy, preferably oxo;
   a mixture of two or more compounds of the formula I, and/or an extract comprising one or more compounds of the formula I, for use in the prophylactic and/or therapeutic treatment of an animal with a (at least preferably) estrogen receptor (ER) related disease or condition;
   where the compounds of the formula I may be present in free form, in the form of a pharmaceutically and/or nutraceutically acceptable salt, in the form of a tautomer, in the form of an ester and/or in the form of a solvate.

Surprisingly aurones of the formula I are found to inhibit binding of estrogen to the estrogen receptor more specifically the named aurones were able to inhibit binding of estrogen to the estrogen receptor beta selectively.

Surprisingly in the roots of Smilax myosotiflora (traditionally named as Ubi Jaga) Aurones were found in a remarkable amount. Some of these compounds were isolated and structurally characterised as shown below. This result is surprisingly new for nearly all species of the genus of Smilax, especially for those naturally grown in South East Asia.

The present invention therefore, in one embodiment, also relates to an extract, especially an extract from Smilax myosotiflora, especially its roots, comprising one or more compounds of the formula I, e.g. in an amount of 10 or more % by weight, e.g. 30 or more % by weight, such as 50 or ore % by weight, for example 80 to 100% by weight.

Further, an optimized alternative procedure for the extract production has been established. There are two special aspects that are addressed with this alternative:
a) the extraction yield of the aurones is strongly dependent on the pH conditions adjusted in the water phase(s) in the extraction process. This is especially crucial in the 1^{st} liquid/liquid separation step.
b) with a second liquid/liquid separation step, which affords again a special pH adjustment, “undesired compounds”, namely homopanthothenic acid, is eliminated quantitatively. Parallel to this elimination, a further enrichment of the aurones has been achieved.

The pH at which the initial extraction and the following 1^{st} liquid/liquid separation step is crucial for the overall yield of the aurones. In a series of experiments the pH of the added water (added to the ethanol) has been adjusted to pH 1, pH 2, pH 3 and pH 4.5. The optimum yield was achieved at pH 2 followed by pH 3 (similar yield), followed by pH 4.5 (50% decrease of yield). In parallel the absolute content of the aurones in the ethyl acetate phases of the 1^{st} liquid/liquid separation step was optimal at pH 2. At pH 1 respectively, no yield or content could be determined since full decomposition of the aurones took place.

Aurones shall be extracted from the plant material under acidic conditions. The preferred pH is in the range of 2-4.5, more preferred pH is 2-3, and the most preferred pH is 2.

The pH conditions in the 2″^{d} liquid/liquid separation step have also been varied to provide opportunity to eliminate “undesired compounds” (homopanthothenic acid), and the pH value of the water phase in the liquid/liquid separation system shall be >7. The preferred pH range is 7-9 and the most preferred pH is 7.4-7.6.

The general expressions, within the present disclosure, preferably have the following or precedingly mentioned meanings, where in each embodiment on, more than one or all more general expressions may, independently of each other, be replaced with the more specific definitions, thus forming preferred embodiments of the invention, respectively.

Where “a compound of the formula I” or “compounds of the formula I” or the like is mentionned, this is intended to include a single compound, a mixture of two or more compounds of the formula I, and/or an extract comprising one or more compounds of the formula I, where the compounds of the formula I may be present in free form, in the form of a pharmaceutically and/or nutraceutically acceptable salt, in the form of a tautomer, in the form of an ester and/or in the form of a solvate.

An estrogen receptor related disease or condition is preferably one that can be partially or completely, permanently or temporarily cured or at least some symptoms of it can be partially or completely, permanently or temporarily, diminished, or removed, or the onset of which can be prophylactically delayed or prevented by binding to estrogen receptor, especially ER-α and/or more especially ER-13, and is especially a disease or disorder or condition selected from the group consisting of: bone loss, bone fractures, osteoporosis, metastatic bone disease, Paget's disease, periodontal disease, hot flashes, cardiovascular disease, restenosis, vascular smooth muscle cell proliferation, obesity, incontinence, multiple sclerosis, sexual dysfunction, hypertension and retinal degeneration. These are preferred diseases or disorders according to the embodiments of the present invention.

Further estrogen receptor related diseases, however, may be selected from the group consisting of impairment of cognitive functioning, age-related mild cognitive impairment, cerebral degenerative disorders, anxiety, depression, perimenopausal depression, post-partum depression, premenstrual syndrome, manic depression, anxiety, dementia, obsessive compulsive behavior, attention deficit disorder, sleep disorders, irritability, impulsivity and anger management.

Preferably, X in formula I is hydrogen and Y is oxo, and if present X′ is hydrogen and Y′ is Preferably, bonds a and c in formula I are double bonds, bonds b and d single bonds, respectively, and, if present, also bonds a′ and c′ are double bonds, bonds b′ and d′ are single bonds.

Among the various possible forms of a compound of the formula I, the free form, the pharmaceutically acceptable salt form and/or the tautomer form are especially preferred.

Tautomers may e.g. be represented by the formulae:

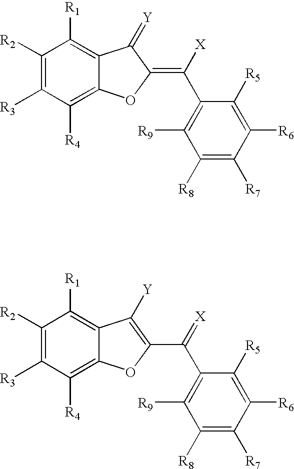

with the meanings as given in claim 1 as appropriate.

“Carbohydrate” refers to a mono or disaccharide consisting of one or two pentoses and/or hexoses optionally in their desoxy forms connected via a glycosidic bond unsubstituted or substituted with one, two, three, four or five substituents independently selected from the group consisting of methyl, ethyl, acetyl, benzoyl or 3,4,5-trihydroxybenzoyl. Examples of preferred pentoses are xylose, arabinose, and either in case when possible in the pyranosidic or furanosidic form. Examples of preferred hexoses are glucose, 6-deoxyglucose, rhamnose, and either in case when possible in the pyranosidic of furanosidic form. Examples of preferred glycosidic connections are 1→4 and 1→6.

Where salt-forming groups (e.g. acidic groups, such as phenolic OH groups) are present within them, a compound of the formula I may be in the free form or in the form of a salt. The term “salt(s)”, as employed herein, denotes basic salts formed with inorganic and/or organic bases. Pharmaceutically (or nutraceutically) acceptable (i.e., non-toxic, physiologically acceptable) salts are preferred, although other salts are also useful, e.g., in isolation or purification steps which may be employed during preparation. Salts of a compound of the formula I may be formed, for example, by reacting a compound of the formula I with an amount of base, such as an equivalent amount, in a medium such as one in which the salt precipitates or in an aqueous medium followed by lyophilization. Also ion exchangers can be used to form salts from free forms or free forms from salts of a compound of the formula I.

A compound of the formula I which contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, N-methyl-D-glucamines, N-methyl-D-glucamides, t-butyl amines, and salts with amino acids such as arginine, lysine and the like. Also salts with salt-forming pharmaceutical and/or nutraceutical carrier materials are possible and encompassed by the invention.

Further, a compound of the formula I (in free form or as salt) may be in the form of a solvate, such as a hydrate.

Where ratios of components are given in %, this means weight %, if not indicated otherwise.

By the term “extract”, either a direct extract (in liquid or preferably dried form), e.g. obtained as described below, or preferably a further enriched extract (obtainable e.g. by one or more further purification steps after extraction, e.g. chromatography, for example as described below) containing one or more, preferably two or more compounds of the formula I is meant.

Preferably, the total weight share of the compound or compounds of the formula I in an extract or mixture of compounds of the formula I or a purified compound of the formula I that is useful according to the invention in the final extract, mixture or compound (direct or further enriched) is in the range from 0.01 to 100% by weight, more preferably from 0.02 to 95%, most preferably 0.05 to 95%, from 0.05 to 50% or e.g. from 0.1 to 90%.

The extracts or compounds according to the invention may be used as such, in the form or pharmaceutical or nutraceutical formulations (the latter term including food additives) or in the form of functional food.

Where a compound or mixture of compounds of the formula I, especially extracts comprising one or more compounds of the formula I, are used as supplement, this means that the compound(s), extract or a pharmaceutical or nutraceutical formulation comprising it or them can be added to any other nutrient or pharmaceutical or nutraceutical, preferably other than (exclude especially mixtures known). Thus they can especially serve as food supplement. However, the compound(s), extract or formulations may also be administered as such.

“Nutraceuticals”, “Functional Food”, or “Functional Food products” (sometimes also called “Foodsceuticals”, “Medicinal Food” or “Designer Food”) for USE according to the present invention are defined as food products (including beverages) suitable for human consumption—the expression comprises any fresh or processed food having a health-promoting and/or disease-preventing property beyond the basic nutritional function of supplying nutrients, including food made from functional food ingredients or fortified with health-promoting additives, especially with effects in the prophylaxis or treatment of a disease or disorder or condition as mentioned herein, that is, a compound of the formula I is used as an ingredient (especially additive) as health benefit agent, especially in an effective amount.

“Comprising” or “including” or “having” wherever used herein is meant not to be limiting to any elements stated subsequently to such term but rather to encompass one or more further elements not specifically mentioned with or without functional importance, that is, the listed steps, elements or options need not be exhaustive. In contrast, “containing” would be used where the elements are limited to those specifically after “containing”.

Where “about” is used or a specific numerical value is given without explicitly mentioning “about”, this preferably means that a given value may deviate to a certain extent from the value given, e.g. preferably by ±20% of the given numerical value, more preferably by ±10% %, e.g. in one embodiment ±5%. Where numerical ranges are given, also where it is not mentioned “about” is present before any numbers.

The functional food products or pharmaceutical products may be manufactured according to any suitable process, preferably comprising extraction of one or more compounds of the formula I and admixing to a functional food product or at least one nutraceutically or pharmaceutically acceptable carrier.

Preferably, a functional food or a pharmaceutical or nutraceutical formulation comprising a compound, more preferably a compound mixture, useful according to the present invention, can be obtained by

(a) extraction of one or more compounds and/or mixture of compounds of the formula I from one or more plants of the genera mentioned below, especially from Smilax myosotiflora (and there especially from the roots); and
(b) mixing the resulting one or more compounds and/or mixtures of compounds as active ingredient in the preparation of the functional food product with the other constituents thereof or in order to obtain a pharmaceutical or nutraceutical formulation with one or more carrier materials or with a solvent, e.g. water or an aqueous solvent (e.g. to give a juice or dispersion or solution).

Further processing steps may precede and/or follow, such as drying (e.g. freeze-drying, spray-drying and evaporation), granulation, agglomeration, concentrating (e.g. to syrups, formed via concentration and/or with the aid of thickeners), pasteurizing, sterilizing, freezing, dissolving, dispersing, filtering, centrifuging, confectioning, and the like.

When one or more compounds and/or a compound mixture or an extract according to the invention are added to a food product or pharmaceutical or nutraceutical, this also results in a functional food product or pharmaceutical or nutraceutical formulation according to the invention.

Preferably, a functional food product according to the invention comprises 0.01 to 30, e.g. 0.02 to 20, such as preferably 0.05 to 5, weight-% of a compound or mixture of compounds of the formula I or of an (especially further enriched) extract according to the invention, the rest being food and/or nutraceutically acceptable carriers and/or customary additives. Further additives may be included, such as vitamins, minerals, e.g. in the form of mineral salts, unsaturated fatty acids or oils or fats comprising them, other extracts, or the like.

The functional food products according to the invention may be of any food type. They may comprise one or more common food ingredients in addition to the food product, such as flavours, fragrances, sugars, fruit, minerals, vitamins, stabilisers, thickeners, dietary fibers, protein, amino acids or the like in appropriate amounts, or mixtures of two or more thereof, in accordance with the desired type of food product.

Examples of basic food products and thus of functional food products according to the invention are fruit or juice products, such as orange and grapefruit, tropical fruits, banana, apple, peach, blackberry, cranberry, plum, prune, apricot, cherry, peer, strawberry, marionberry, black currant, red currant, tomato, vegetable, e.g. carrot, or blueberry juice, soy-based beverages, or concentrates thereof, respectively; lemonades; extracts, e.g. coffee, tea, green tea; dairy type products, such as milk, dairy spreads, quark, cheese, cream cheese, custards, puddings, mousses, milk type drinks and yoghurt; frozen confectionary products, such as ice-cream, frozen yoghurt, sorbet, ice milk, frozen custard, water-ices, granitas and frozen fruit purees; baked goods, such as bread, cakes, biscuits, cookies or crackers; spreads, e.g. margarine, butter, peanut butter honey; snacks, e.g. chocolate bars, muesli bars; pasta products or other cereal products, such as muesli; ready-to-serve-dishes; frozen food; tinned food; syrups; oils, such as salad oil; sauces, such as salad dressings, mayonnaise; fillings; dips; chewing gums; sherbet; spices; cooking salt; instant drink powders, such as instant coffee, instant tee or instant cocoa powder; instant powders e.g. for pudding or other desserts; or the like.

One or more other customary additives may be present, such as flavour, fragrances or other additives, such as one or more selected from stabilizers, e.g. thickeners; colouring agents, such as edible pigments or food dyes; bulking agents, such as fruit pulp, e.g. in dried form; polyols, such as xylitol, mannitol, maltitol or the like; preservatives, such as sodium or potassium benzoate, sodium or calcium carbonate or other food grade preservatives; antioxidants, such as ascorbic acid, carotionoids, tocopherols or polyphenols; mono-, oligo- or polysaccharides, such as glucose, fructose, sucrose, soy-oligosaccharides, xylo-oligosaccharides, galacto-oligosaccharides; other artificial or natural non- or low-caloric sweeteners, such as aspartame or acesulfame; bitterness blockers; acidifiers in the form of edible acids, such as citric acids, acetic acid, lactic acid, adipic acid; flavours, e.g. artificial or natural (e.g. botanical flavours); emulsifiers; thiols, e.g. allylic thiols; diluents, e.g. maltodextrose; wetting agents, e.g. glycerol; stabilizers; coatings; isotonic agents; absorption promoting or delaying agents; and/or the like.

The one or more compounds of the formula I or compound mixtures thereof or extracts comprising them according to the invention can also be comprised in confectioned formulations to be added to foods including beverages, e.g. in the form of powders or granules, e.g. freeze-dried or spray-dried, concentrates, solutions, dispersions or other instant form, or the like.

The pharmaceutical or nutraceutical formulation (=compositions) according to the present invention can be prepared in various forms, such as granules, tablets, pills, syrups, solutions, dispersions, suppositories, capsules, suspensions, salves, lotions and the like. Pharmaceutical grade or food grade organic or inorganic carriers and/or diluents suitable for oral and topical use can be used to formulate compositions containing the therapeutically-active compounds. Diluents known in the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The compositions may also include one or more of the following: carrier proteins such as serum albumin; buffers; fillers such as microcrystalline cellulose, lactose, corn and other starches; binding agents; sweeteners and other flavouring or fragrancing agents; coloring agents; and polyethylene glycol. Those additives are well known in the art, and are used in a variety of formulations.

By “administered” herein is meant administration of a prophylactically and/or therapeutically effective dose of a compound of the formula I or a mixture of compounds of the formula I, or an extract comprising one or more of them, to an animal, especially a patient. By “therapeutically effective dose” herein is meant a dose that produces the effects for which it is administered, especially an ameliorative or therapeutic effect on ER dependent diseases or conditions of the central nervous system, more especially on Parkinson's Disease, and dementia.

An animal or human, especially being a “patient” or “subject” for the purposes of the present invention, includes especially humans and further other (especially warm-blooded) animals. Thus, the compound of the formula I or a mixture of compounds of the formula I, or an extract comprising one or more of them, are applicable to both humans and animals. In the preferred embodiment the patient is a human. The patients will be treated either in prophylactic or therapeutic intention.

Typically, the compound of the formula I or a mixture of compounds of the formula I, or an extract comprising one or more of them, having therapeutical activity mentioned hereinbefore may be administered with at least one physiologically (=pharmaceutically or nutraceutically) acceptable carrier to a patient, as described herein. The total concentration of therapeutically active compound of the formula I or a mixture of compounds of the formula I or extracts comprising them in the formulation, as well as in a pharmaceutical formulation as such according to the invention, may vary from about 0.001-100 wt %, e.g. from 0.1 to 50% by weight, the rest being the carrier material(s) and/or customary additives.

The compound of the formula I or a mixture of compounds of the formula I or extracts comprising them may be administered alone or in combination with other treatments, including drugs, i.e., other estrogen receptor modulators like, as example which are not limiting, alcobifene or its precursor 4-[7-(2,2-dimethyl-1-oxopropoxy)-4-methyl-2-[4-[2-(1-piperidynyl)ethoxy]phenyl]-2H-1-benzopyran-3-yl]-phenyl-2,2-dimethylpropanoate, estrogen, progestogen, estradiol, raloxifene, lasofoxidene, TSE-424, tamoxifen, idoxifene, LY353381, LY117081, toremifene, fulvestrant, 4,4′-dihydroxybenzophenone-2,4-dinitrophenyl-hydrazone, and SH646.

“Combination” does not necessarily mean a fixed combination but may also mean that the compound(s) of the formula I or the extract comprising it or them may be administered in a chronically staggered manner with the combination partner(s), e.g. in the form of a kit of parts (which also is an embodiment of the invention) with other combination partners, other than those excluded hereinbefore. Preferably, the chronically staggered administration takes place such that the combination partners mutually influence, especially intensify (e.g. by way of an additive or preferably synergistic effect) their therapeutic efficiency.

Other helpful drugs or active agents may be administered, e.g. psychoactive agents, agents that help in the treatment of addictive behaviour, e.g. nicotine addiction, or the like, especially in so far as they help to support the prophylaxis or treatment according to the invention intended.

The dosage in both nutraceutical or pharmaceutical use typically is such that the amount of the compound(s) of the formula I administered to a patient is such that it is effective in modulating the estrogen receptor, or preferably a daily dose of about 0.2 to 1000 g, e.g. 0.5 to 5 g is administered to a person with a weight of 70 kg per day in one or more, e.g. 1 to 3, dosages (children or persons with differing weights receive a correspondingly modified dosage).

Extracts comprising one or more compounds of the formula I can be prepared from plants as mentioned above or below or plant parts.

The following list provides possible sources of compounds of the formula I or extracts comprising them:

### 1. List of all Smilax:

S. aberrans, S. acanthophylla, S. aculeata, S. aculeatissima, S. acuminata, S. acutifolia, S. adhaerens, S. aequatorialis, S. alba, S. alpini, S. altissima, S. amaurophlebia, S. amblyobasis, S. ampla, S. anamitica, S. anceps, S. anguina, S. angustifolia, S. annamensis, S. annua, S. argyraea, S. argyrea, S. arisanensis, S. aristolochiaefolia, S. aristolochiifolia, S. asparagoides, S. aspera, S. aspero-variabilis, S. astrosperma, S. auraimensis, S. auriculata, S. australis, S. austrosinensis, S. austrozhejiangensis, S. balansaeana, S. balbisiana, S. balearica, S. banglaoensis, S. bapouensis, S. barbata, S. barbillana, S. basilata, S. bauhinioides, S. bella, S. benthamiana, S. bermudensis, S. bernhardi, S. berteroi, S. beyrichii, S. biflora, S. biltmoreana, S. biumbellata, S. blancoi, S. blinii, S. blumei, S. bockii, S. bodinieri, S. bona-nox, S. bonii, S. boninensis, S. borbonica, S. borneensis, S. botteri, S. botterii, S. brasiliensis, S. brevipes, S. caduca, S. calaris, S. califormica, S. calocardia, S. calophylla, S. cambodiana, S. campestris, S. canaliculata, S. canariensis, S. candelariae, S. canellaefolia, S. capitata, S. castaneiflora, S. catalonica, S. caudata, S. cavaleriei, S. celebica, S. cercidifolia, S. ceylanica, S. chapaensis, S. chiapensis, S. chimantensis, S. china, S. chingii, S. chiriquensis, S. ciliata, S. cinerea, S. cinnamomes, S. cinnamomifolia, S. cinnamomiifolia, S. cinnamommea, S. cissoides, S. cocculoides, S. cognata, S. collina, S. colossea, S. colubrina, S. columnifera, S. compressa, S. conchipes, S. conferta, S. corbularia, S. corcovadensis, S. cordato-ovata, S. cordifolia, S. coriacea, S. coriifolia, S. cumanensis, S. cuspidata, S. cyclophylla, S. cynanchifolia, S. cynodon, S. darrisii, S. davidiana, S. decipiens, S. deltifolia, S. densibarbata, S. densiflora, S. dentata, S. dilatata, S. discolor, S. discotis, S. divaricata, S. diversifolia, S. domingensis, S. dominguensis, S. duidae, S. dulcis, S. dunniana, S. ecirrata, S. ecirrhata, S. ehrenbergiana, S. elastica, S. elegans, S. elegantissima, S. elliptica, S. elmeri, S. elongato-reticulata, S. elongato-umbellata, S. emeiensis, S. engelmanniana, S. engleriana, S. erythrocarpa, S. eucalyptifolia, S. excelsa, S. extensa, S. farinosa, S. febrifuga, S. ferox, S. ficifolia, S. fistulosa, S. flaccida, S. flavescens, S. flavicaulis, S. flexuosa, S. floribunda, S. fluminensis, S. fokiensis, S. fooningensis, S. formosana, S. fulgens, S. gagnepainii, S. gaudichaudiana, S. gaultheriifolia, S. gaumeri, S. gaumerii, S. gemina, S. gentlei, S. gigantocarpa, S. gilva, S. glabra, S. glauca, S. glaucocarpos, S. glaucochina, S. glauco-china, S. glaucophylla, S. globifera, S. globulifera, S. glyciphylla, S. glycyphylla, S. goetzeana, S. goudotiana, S. goyazana, S. graciliflora, S. gracillima, S. grandiflora, S. grandifolia, S. grandis, S. griffithii, S. guianensis, S. guiyangensis, S. gymnopoda, S. hastata, S. havanensis, S. hawaiensis, S. hayatae, S. hederaefolia, S. helferi, S. hemsleyana, S. herbacea, S. heterophylla, S. higoensis, S. hilariana, S. hirsutior, S. hohenackeri, S. hongkongensis, S. hookeri, S. horrida, S. horridiramula, S. hostmanniana, S. hugeri, S. humilis, S. hypoglauca, S. ilicifolia, S. illinoensis, S. immersa, S. impressinervia, S. incerta, S. indica, S. indosinica, S. inermis, S. insignis, S. intricatissima, S. invenusta, S. inversa, S. iriomotensis, S. irrogata, S. irrorata, S. jacquini, S. jacquinii, S. jalapensis, S. jamesii, S. japicanga, S. japonica, S. jauaensis, S. javensis, S. jiankunii, S. kainantensis, S. kaniensis, S. kerberi, S. keyensis, S. klotzschii, S. korthalsii, S. kraussiana, S. krukovii, S. kunthii, S. kwangsiensis, S. labidurommae, S. labordei, S. laevis, S. lamarensis, S. lancaefolia, S. lanceaefolia, S. lanceifolia, S. lancifolia, S. lappacea, S. larvata, S. lasioneura, S. lasioneuron, S. lasseriana, S. lata, S. latifolia, S. latipes, S. laurifolia, S. laurina, S. lebrunii, S. lessertiana, S. leucocarpa, S. leucophylla, S. ligustrifolia, S. liukiuensis, S. loheri, S. lomoplis, S. longebracteolata, S. longifolia, S. longipedunculata, S. longipes, S. loupouensis, S. luculenta, S. lundellii, S. lunglingensis, S. lushuiensis, S. luteocaulis, S. lutescens, S. luzonensis, S. macabucha, S. macalucha, S. maclurei, S. macrocarpa, S. macrophylla, S. macropoda, S. maculata, S. magnifolia, S. mairei, S. malipoensis, S. marginata, S. marginulata, S. maritima, S. martini, S. mauritanica, S. maximowiczii, S. maypurensis, S. mazatlanensis, S. mcclurei, S. medica, S. medicinalis, S. megacarpa, S. megalantha, S. megalophylla, S. melanocarpa, S. melastomifolia, S. membranacea, S. mengmaensis, S. menispermoidea, S. mexicana, S. micro-china, S. microphylla, S. micropoda, S. microscola, S. milleri, S. minarum, S. minutiflora, S. modesta, S. mollis, S. montana, S. montevidensis, S. moranensis, S. morongii, S. morsaniana, S. mossambicensis, S. multiflora, S. munda, S. munita, S. muricata, S. muscosa, S. myosotiflora, S. myrtillus, S. nageliana, S. nana, S. nantoensis, S. narcotica, S. nebelii, S. neocaledonica, S. neo-calcdonica, S. nervo-marginata, S. nigra, S. nigrescans, S. nipponica, S. nitida, S. nova-guineensis, S. obliqua, S. obliquata, S. oblonga, S. oblongata, S. oblongifolia, S. obtusa, S. occidentalis, S. ocreata, S. odontolama, S. odontoloma, S. odoratissima, S. officinalis, S. oldhami, S. oldhamii, S. opaca, S. orbiculata, S. ornata, S. orthoptera, S. osmastonii, S. outanscianensis, S. ovalifolia, S. ovata, S. ovatolanceolata, S. ovato-rotunda, S. oxycarpa, S. oxyphylla, S. pachysandroides, S. pallescens, S. panamensis, S. pandurata, S. panduriformis, S. paniculata, S. papuana, S. papyracea, S. parviflora, S. parvifolia, S. pavoniana, S. peduncularis, S. peguana, S. pekingensis, S. pendulina, S. perfoliata, S. pertenuis, S. perulata, S. petelotii, S. petiolatumida, S. phyllantha, S. phyllobola, S. picta, S. pilcomayensis, S. pilosa, S. pinfaensis, S. pirarensis, S. pittieriana, S. plani-peduncula, S. planipes, S. platoplis, S. platycentron, S. platyphylla, S. plurifurcata, S. poeppigii, S. pohliana, S. poilanei, S. poiretii, S. polyantha, S. polycephala, S. polycolea, S. populnea, S. pottingeri, S. pringlei, S. procera, S. prolifera, S. pruinosa, S. pseudochina, S. pseudo-china, S. pseudo-sarsa, S. pseudosyphilitica, S. pseudo-syphilitica, S. pteropus, S. pubens, S. pubera, S. puberula, S. pulverulenta, S. pumila, S. purampui, S. purhampuy, S. purpurata, S. purpusii, S. purtrampui, S. pygmaea, S. quadrangularis, S. quadrangulata, S. quadrata, S. quadrumbellata, S. quinquenervia, S. ramiflora, S. ramonensis, S. randaiensis, S. regelii, S. remotinervis, S. renifolia, S. reniformis, S. reticulata, S. retroflexa, S. rettiana, S. retusa, S. rhombifolia, S. riedeliana, S. rigida, S. riparia, S. ripogonum, S. ripponica, S. robert-kingii, S. robusta, S. rotundiflora, S. rotundifolia, S. roxburghiana, S. roxburghii, S. rubiginosa, S. rubra, S. rubriflora, S. rubromarginata, S. rufa, S. rufescens, S. ruizana, S. ruiziana, S. sadoensis, S. sagittaefolia, S. sagittata, S. sagittifera, S. sagittifolia, S. salicifolia, S. salutaris, S. sanguinea, S. santaremensis, S. sarsaparilla, S. sarumame, S. saulensis, S. saxicola, S. scabriuscula, S. scalaris, S. schaffneriana, S. schafneriana, S. schiedeana, S. schlechtendalii, S. schombiurgkiana, S. schomburgkiana, S. scobinicaulis, S. sebeana, S. selloana, S. semiamplexicaulis, S. sempervirens, S. senticosa, S. setiramula, S. setosa, S. shuttleworthii, S. siamensis, S. siderophylla, S. sieboldi, S. sieboldii, S. simadai, S. simulans, S. sinclairi, S. singaporensis, S. siphilitica, S. smalli, S. smallii, S. solanifolia, S. spicata, S. spinescens, S. spinosa, S. spinulosa, S. spissa, S. sprengelii, S. spruceana, S. staminea, S. standleyi, S. stans, S. stemonifolia, S. stenophylla, S. stipulacea, S. subaculeata, S. subarmata, S. subpubescens, S. subsessiliflora, S. surinamensis, S. sylvatica, S. synandra, S. syphilitica, S. syringoides, S. taiheiensis, S. takaoensis, S. talbotiana, S. tamnoides, S. taquetii, S. telfaireana, S. tenuis, S. tenuissima, S. tetragona, S. tetraptera, S. thomsoniana, S. tijucensis, S. timorensis, S. tomentosa, S. tonduzii, S. tongaensis, S. tortopetiolata, S. tortuosa, S. trachyclada, S. trachypoda, S. trifurcata, S. trigona, S. trinervula, S. trukensis, S. tsaii, S. tsinchengshanensis, S. tuberculata, S. turbans, S. umbellata, S. umbellifera, S. umbrosa, S. undulata, S. uruapensis, S. utilis, S. vaga, S. vaginata, S. vanchingshanensis, S. vanilliodora, S. variabilis, S. variegata, S. velutina, S. venosa, S. verticalis, S. vicaria, S. villandia, S. viminea, S. virginiana, S. viscifolia, S. vitiensis, S. wagneriana, S. wallichii, S. walteri, S. watsonii, S. wightii, S. williamsi, S. williamsii, S. willkommii, S. woodii, S. xalapensis, S. yai, S. yui, S. yunnanensis, S. zeylanica, S. zollingeri, S. polyacantha, S. gigantea, S. kingii.

This includes plants with traditional names such as Sarsaparilla, Greenbriar, Catbriar, Horsebriar, Bullbirar, Ubi Jaga, Ubi Besi, Akar Ali, Akar Ding, Akar Tanding, Akar Restong, Kerating or Manto.

Preferred are Smilax varieties that can be found in Southeast Asia:

### 2. List of Smilax in SE Asia:

S. blumei, S. calophylla, S. china, S. corbularia, S. extensa, S. gigantea, S. glabra, S. helferi, S. kingii, S. laevis, S. lanceifolia, S. leucophylla, S. luzonensis, S. macrocarpa, S. megacarpa, S. myosotiflora, S. polyacantha, S. verticalis, S. walteri, S. woodii, S. zeylanica

Ubi Jaga, Ubi Besi, Akar Ali, Akar Ding, Akar Tanding, Akar Restong, Kerating, Manto, Akar Dedingin, Itah Besi, Keleh, Ali Bertinggong (which are all synonyms for Smilax myosotiflora), Akar dawai, dawai dawai, sedawai, akar kancil (which are all synonyms for Smilax calophylla), Radix Chinae, China Root, Gadong China, Gadong Saberang, Akar Restong, Ubat Raja, Akar Resting, Chinese Sarsaparilla, Peundang (which are all synonyms for Smilax china and Smilax ferox), Ubi Danau or Danai, Akar Banar, Channar Bokor, Sarsaparillang-Puti, Banag, Kaguno, Wanabekira (which are all synonyms for Smilax leucophylla), Akar Banar, Banar Babi, Chanar Babi, Akar Kelona Betina, Semenjoh, Akar Gadong Tikus, Gadong Jantan (which are all synonyms for Smilax helferi and Smilax luzonensis), Akar Kelona, Akar Banar, Akar Rebanar, Akar Beruboh, Akar Lampu Bukit, Chanar Bokor, Chanar Gede, Chanar Gengge, Chanar Minyak (which are all synonyms for Smilax megacarpa), Canar Bokor, Canar Gede, Canar Minyak (which are all synonyms for Smilax macrocarpa), Koh Kong, Xieng Khouang, Hua Khaao-yen wok (which are all synonyms for Smilax corbularia), Koh Kong, Yaa Hua (which are all synonyms for Smilax glabra), Dao, Naam Dao, Thao Yang Dong, Kim Cang (which are all synonyms for Smilax lanceifolia), Akar Gadung Tikus, Khueang, Yaan That, Faa Laep (which are all synonyms for Smilax luzonensis), Voe Me, Khrua Daao, Kim Chang (which are all synonyms for Smilax verticalis and Smilax simulans), Kayu Cina Utan, Saihe Maruani, Asaihe Tuni (which are all synonyms for Smilax zeylanica).

Especially preferred is:

3. Smilax myosotiflora or Ubi Jaga.

Plant parts are, e.g., leaves, bark, flowers, buds, fruits, stems, shoots, roots, tubers or other parts of plants, and they or the plants can be complete, hackled, crushed, chopped up, broken up, homogenized, dried, fermented or treated otherwise.

A compound the formula I or a mixture of compounds of the formula I, or an extract comprising one or more of them, of the present invention can be prepared by extracting and preferably enriching up to isolating them from the plants or parts of plants. Auxiliary means such as (especially ultrasonic) sonication, heating (e.g. to temperatures from room temperature to 50° C.), stirring, re-extraction, evaporation or the like, may be used to allow for appropriate extraction.

Extraction preferably takes place with a non polar or more preferably a polar solvent or solvent mixture, e.g. water and/or an alcohol, such as ethanol, and/or with a liquid gas, especially superfluid CO₂.

Preferably, the extracts can subsequently be further enriched by one or more additional purification steps, such as distribution (especially into an apolar solvent, such as an alkane and/or an ester, e.g. n-heptane and ethyl acetate), precipitation (e.g. crystallisation) or chromatography, by which it is possible to obtain further enriched extracts or isolated compounds of the formula I.

In order to optimize the production of the compound of the formula I, e.g. aurones, after one or more extraction steps, a liquid-liquid extraction procedure can be employed. Liquid-liquid extraction, also known as solvent extraction or solvent partitioning, is a method to separate compounds based on their relative solubilities in two different immiscible liquids, preferably not or only partially miscible, usually water and an organic solvent. This way a desired substance or substance mixture can be extracted from one first liquid phase into another liquid phase or remain in the first phase, while less desired substances remain in the other phase, respectively. It is also possible to influence the distribution by establishing specific conditions in the solvents used for partition, such as acidic, neutral or basis conditions. Thus, e.g., less polar molecules or polar neutralized acids or basis can be induced to distribute into the less polar solvent, charged or otherwise polar molecules, such as the dissociated acids or bases preferably can be directed into the more polar solvent. Liquid-liquid extraction is a basic technique in chemical laboratories, where it is preferably performed using a separatory funnel. For the enrichment of phytochemicals from a crude plant extract, usually the concentrated extract, is partly dissolved in water or solvent-containing water (solvents here are co-solvents, for example methanol, ethanol, propanol, isopropanol, acetone, acetonitrile or other water-miscible solvents) and extracted once or successively with identical or different water-immiscible solvents or solvent mixtures, preferably not or only partially miscible, in the case of successively used different water-immiscible or partially water-miscible solvents, successively using solvents of e.g. increasing polarity (for example, without that this is intended to exclude other alternatives known to the person skilled in the art, in the order of; 1. heptane, hexane, pentane, cyclohexane, petroleum ether; 2. diethyl ether, toluene, benzene, t-butyl methyl ether, chloroform, dichloromethane, ethyl methyl ketone, dioxane, tetrahydrofuran; 3. ethyl acetate).

Further, surprisingly it could be shown that an improved yield can be obtained when an extraction and purification process is used that avoids strongly alkaline (e.g. pH 9 or larger) and strongly acidic conditions (e.g. pH 1.8 or lower) conditions—without being bound to this theory, a possible explanation may be that the compounds of the formula I might be prone to degradation, such as hydrolysis under too alkaline conditions.

Therefore a preferred procedure for the extract production has been found that, in particular, addresses various specific aspects:
a) the extraction yield of the aurones is strongly dependent on the pH conditions adjusted in the water phase(s) in the extraction process. This is especially important and thus preferred in a first liquid/liquid separation step;
b) with a second liquid/liquid separation step, which preferably again comprises a specific pH adjustment, “undesired compounds”, such as homopanthothenic acid, are eliminated to a wide extent, e.g. in the case of homopanthothenic acid even quantitatively. This allows to reduce or eliminate undesired components and thus to diminish e.g. the risk of undesired side effects or toxic components.
c) Parallel to the elimination of undesired material found after the first and the second extraction step, a further enrichment of the compounds of the formula I (and thus aurones) has been achieved.

Especially the adjustment of the pH at which the initial extraction and the following first liquid/liquid separation step has surprisingly been found to be of high importance for the overall yield of the compounds of the formula I (aurones). In a series of experiments, the pH of the added water (added to the ethanol) has been adjusted to various pH values, e.g. to pH 1, pH 2, pH 3 and pH 4.5. The optimum yield was achieved at about pH 2 followed by about pH 3 (similar yield), followed by about pH 4.5 (50% decrease of yield). In parallel the absolute content of the aurones in the ethyl acetate phases of the first liquid/liquid separation step was found to be highest at about pH 2. At pH 1 respectively, no yield or content could be determined, presumably and according to analytical results since virtually complete decomposition of the aurones took place.

The compounds of the formula I, e.g. Aurones therefore preferably are extracted from the plant material (e.g. S. myosotiflora) and subjected to a first liquid/liquid extraction under acidic conditions, respectively, which is what a preferred embodiment of the extraction and purification process according to the invention comprises. The preferred pH is in the range of about 2 to about 4.5, more preferred pH is about 2 to about 3, and the most preferred pH is about 2. The aurones/compounds of the formula I are here enriched preferably in the less polar solvent phase.

The pH conditions in a subsequent second liquid/liquid separation step have also been varied to provide opportunity to eliminate “undesired compounds” (such as homopanthothenic acid), and the pH value of the water phase in the liquid/liquid separation system has been found to be preferably about neutral to slightly alkaline, e.g. about 7 or larger. A preferred pH range is about 7 to about 9, and a most preferred pH is 7.4 to 7.6. The aurones/compounds of the formula I are here enriched preferably in the less polar solvent phase.

Thus, in one aspect the present invention also relates to an extraction and purification (or at least enrichment) process comprising an extraction step from a plant or plant parts and a first liquid/liquid separation step under acidic conditions, respectively, as described above or below, and a subsequent second liquid/liquid extraction of the material found in the less polar phase of the first extraction step, preferably under neutral to slightly alkaline conditions mentioned above or e.g. in the examples, in particular as mentioned to be preferred, yielding a purified product from the less polar phase also in the second extraction step. Further liquid/liquid partition or other purification may follow and can lead to yet more pure product.

Alternatively, further purification to yield enriched mixtures of few compounds of the formula I or pure compounds of the formula I is added, e.g. by chromatographic methods, e.g. as shown in the Examples.

Where “useful” is mentioned, this especially refers to one or more of the following embodiments of the invention which can be inserted wherever “useful” is mentioned:

(1) A compound of the formula I, or a mixture of compounds of the formula I, or especially a (preferably further enriched) extract comprising one or more compounds of the formula I, for use in therapeutic (including prophylactic) treatment of an animal, preferably a mammal, especially a human, for the treatment of an estrogen receptor (ER) related disease or disorder or condition, especially a disease or disorder or condition mentioned as preferred.
(2) A pharmaceutical or nutraceutical composition comprising a compound of the formula I, or a mixture of compounds of the formula I, or especially a (preferably further enriched) extract comprising one or more compounds of the formula I, as active ingredient together with a pharmaceutically acceptable diluent or carrier, especially for use in the therapeutic and/or prophylactic treatment mentioned under (1).
(2′) A pharmaceutical or nutraceutical composition for the treatment as mentioned under (1) comprising a compound of the formula I, or a mixture of compounds of the formula I, or especially a (preferably further enriched) extract comprising one or more compounds of the formula I, and a pharmaceutically acceptable diluent or carrier, as active ingredient supplement to a food.
(3) A functional food comprising a compound of the formula I, or a mixture of compounds of the formula I, or especially a (preferably further enriched) extract, as active ingredient for the treatment as mentioned under (1).
(4) A method for the treatment as mentioned under (1), comprising administering a pharmaceutically or nutraceutically effective amount of a compound of the formula I, a mixture of compounds of the formula I, or a (preferably further enriched) extract comprising one or more compounds of the formula I, as active ingredient, especially to an individual in need thereof.
(5) The use of a compound of the formula I, or a mixture of compounds of the formula I, or a (preferably further enriched) extract comprising one or more compounds of the formula I, as active ingredient for the manufacture of a medicament or nutraceutical or food supplement for the treatment mentioned under (1).
(6) A method or use as defined under (4), comprising co-administration, e.g. concomitantly or in sequence, of a therapeutically effective amount of compound of the formula I, or a mixture of compounds of the formula I, or a (preferably further enriched) extract comprising one or more compounds of the formula I, as active ingredient and a different pharmaceutically active compound and/or a pharmaceutically acceptable salt thereof, said different pharmaceutically active compound and/or salt thereof being especially for use in the treatment as mentioned under (1).
(7) A combination product comprising a therapeutically effective amount of a compound of the formula I, or a mixture of compounds of the formula I, or a (preferably further enriched) extract comprising one or more compounds of the formula I, as active ingredient, and a different pharmaceutically active compound and/or a pharmaceutically acceptable salt thereof, said second pharmaceutically active compound being especially for use or of use in the treatment mentioned under (1).

For any of the uses, the use is such that the compound(s) of formula I or the extract comprising such compound(s) of the formula I are the active ingredient, that is, they are already alone capable of achieving the intended effect.

By “administering” herein is especially meant administration of a therapeutically effective dose of a compound of the formula I, or a mixture of compounds of the formula I, to a cell either in cell culture or especially to an animal, especially a human patient. By “therapeutically effective dose” herein is preferably meant a dose that produces the effects for which it is administered.

The pharmaceutical or nutraceutical preparations may be sterilized and/or may contain carrier materials or adjuvants such as preservatives, stabilizers, binders, disintegrants, wetting agents, skin or mucous membrane penetration enhancers, emulsifiers, salts for varying the osmotic pressure and/or buffers, or other ingredients, excipients or carrier materials known in the art.

### BRIEF DESCRIPTION OF THE FIGURES

FIG. 1: workflow diagram, isolation procedure.

FIG. 2: HPLC-UV-MS-ELSD analysis of an Ethyl acetate extract of Smilax myosotifolia.

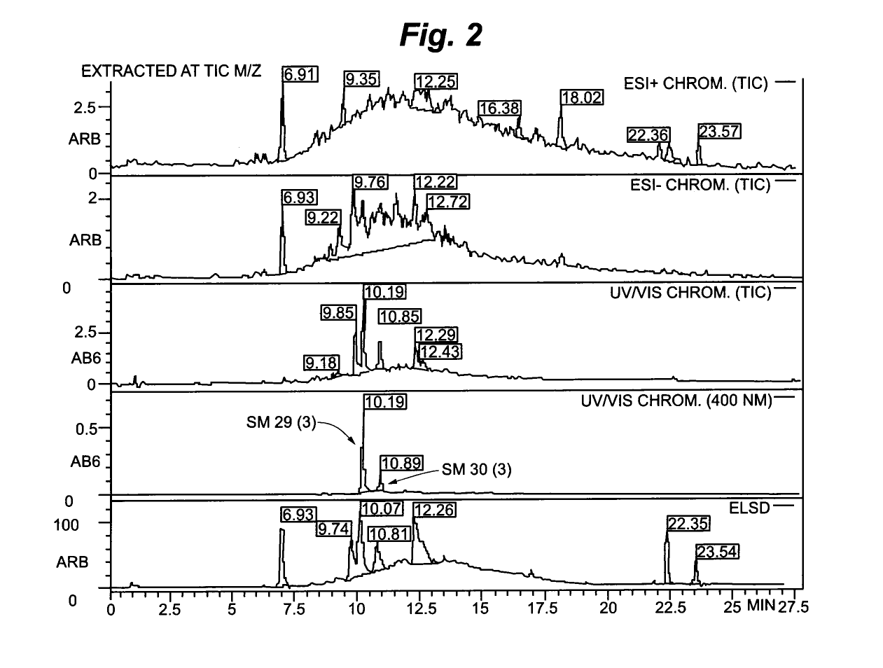

FIG. 3: Typical UV spectrum of an aurone.

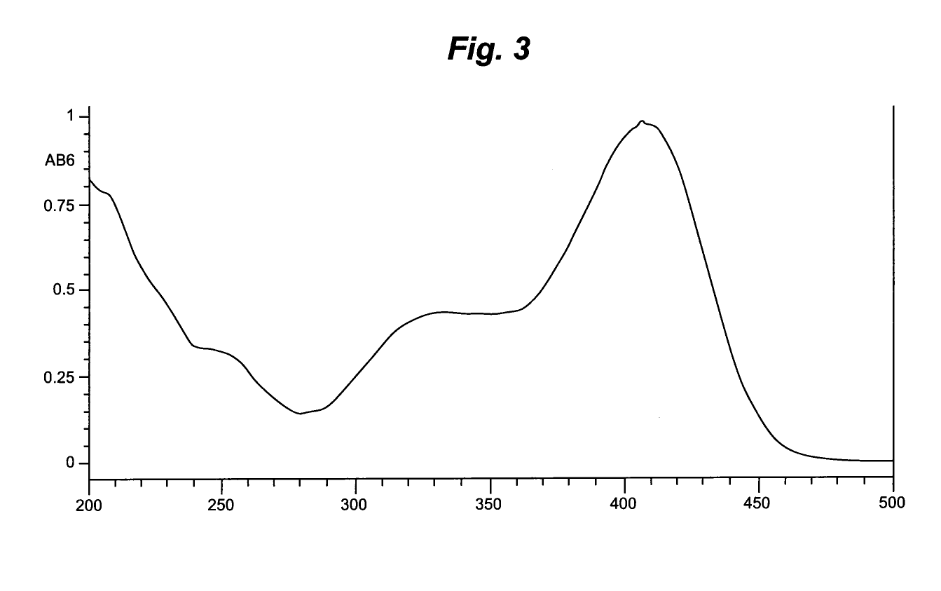

FIG. 4: ¹H NMR spectrum of SM 29 (3) (DMSO-d₆, 500 MHz).

FIG. 5: ¹H NMR spectrum of SM 30 (3) (DMSO-d₆, 500 MHz).

### PREFERRED EMBODIMENTS

A compound of the formula I, e.g. the compound, compound mixture or an extract comprising one or more compounds of the formula I is preferably useful in the treatment of a disease as mentioned that relates to (especially insufficient activity of) the estrogen receptor (ER), more preferably on the (especially insufficient activity of) the estrogen receptor beta (Erbeta or ER-β).

Whether a compound is effective here is defined as follows: It shows ER activation in at least one of the assays as shown below in the Examples.

A disease or disorder or condition “related to estrogen receptor” is thus preferably a disease or disorder that can be partially or completely, permanently or temporarily cured, or at least some symptoms of which can be partially or completely, permanently or temporarily, diminished, or removed, or the onset of which can be prophylactically delayed or prevented by (at least partial) activation of estrogen receptor, especially ERα and/or more especially ERβ. “Related to ER” thus also means that ER modulation contributes to amelioration or even cures regarding the symptoms of the disease, thus preferably meaning “responding” to ER modulation. In particular such a disease or disorder or condition related to estrogen receptor is one where at 10 μM concentration in the test systems given in the Examples a compound of the formula I shows an inhibition of estrogen binding to Erβ that is at least 3-fold, preferably at least about 5-fold stronger than that to Era.

An embodiment of the invention related to a method of treating or preventing Paget's disease, postmenopausal osteoporosis, glucocorticoid osteoporosis, hypocalcaemia of malignancy, bone loss and bone fractures in a mammal, especially a human, in need of such treatment, comprising administering to the mammal a therapeutically effective amount of any of the compounds or nutraceutical or pharmaceutical compositions described above.

Another embodiment of the invention relates to a method of treating of preventing periodontal disease or tooth loss in a mammal in need thereof, comprising administering to the mammal a therapeutically effective amount of any of the compounds or nutraceutical or pharmaceutical compositions described above.

Another embodiment of the invention relates to a method of treating or preventing cardiovascular disease, restenosis, lowering levels of LDL cholesterol and inhibiting vascular smooth muscle cell proliferation in a mammal in need thereof, comprising administering to the mammal a therapeutically effective amount of any of the compounds or nutraceutical or pharmaceutical compositions described above.

Another embodiment of the invention is a method of treating or preventing hypertension, comprising administering to the mammal a therapeutically effective amount of any of the compounds or nutraceutical or pharmaceutical compositions described above.

Another embodiment of the invention relates to a method of treating or preventing obesity in a mammal in need thereof, comprising administering to the mammal a therapeutically effective amount of any of the compounds or nutraceutical or pharmaceutical compositions described above.

Another embodiment of the invention relates to a method of treating or preventing the impairment of cognitive functioning, age-related mild cognitive impairment or cerebral degenerative disorders, such multiple sclerosis, age related dementia or dementia in general in a mammal in need thereof, comprising administering to the mammal a therapeutically effective amount of any of the compounds or pharmaceutical compositions described above.

Another embodiment of the invention relates to a method of treating or preventing sexual dysfunction in males or females comprising administering to the mammal a therapeutically effective amount of any of the compounds or pharmaceutical compositions described above.

Another embodiment of the invention relates to a method of treating or preventing retinal degeneration comprising administering to the mammal a therapeutically effective amount of any of the compounds or pharmaceutical compositions described above.

A compound of the formula I may be used alone, or in combination with one or more additional neurogenic agents. Among the treatment goals are also improvement and (eg. prophylactic) support of cognitive function as well as neuroprotection in diseases states.

Preferably, a compound of the formula I is a natural compound, that is, a compound that is present in and can be isolated or extracted from natural sources (especially those mentioned in detail) without chemical synthesis steps (though it may also be prepared by chemical synthesis), and not a derivative only obtainable by chemical synthesis.

Preferred are also the embodiments of the invention given in the claims, which are incorporated into the present description by reference, and especially in the examples.

In yet another alternative, the present invention relates to an extract from Smilax myosotiflora, especially from its roots, comprising a compound of the formula I described above without the proviso, and embodiments claiming a usefulness as described above, especially for use in the treatment of a disease or disorder related to estrogen receptor.

In a more preferred embodiment, also the proviso given under formula I may be present in the embodiments of the last paragraph that if R₁, R₃ and R₇ each are hydroxyl, R₂, R₄, R₅ and R₉ each are hydrogen and one of R₆ and R₈ is bound via an oxygen, then the other of R₆ and R₈ has one of the meanings mentioned above other than H.

To clarify, the present invention preferably does not relate to the use of compounds of the formula

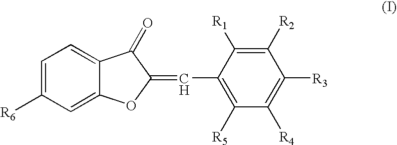

as defined in JP H10-209268 as inhibitors of 17β-hydroxysteroid-dehydrogenase used as a preventive and therapeutic of prostatic cancer, prostatomegaly, masculinism, breast cancer, mastopathy, uterine cancer, endometriosis, and ovarian cancer, where the disease is not ER dependent, or more preferably of said diseases in general with said compounds.

Also to clarify, the present invention preferably does in addition not relate to the use of compounds of the formula

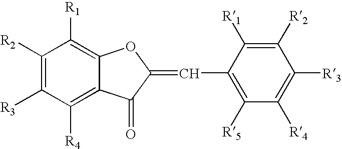

as defined in WO1991/017749 as inhibitors of type II ER used as an preventive and therapeutic of the growth of proliferating cells, autoimmune diseases and cancer, especially breast cancer, prostatic hyperplasia, cervical hyperplasia, uterine hyperplasia and endometriosis, where the disease is not classical ER dependent, or more preferably of said diseases in general with said compounds.

Patent applications and other references, where mentioned, are included herein by reference, especially regarding the passages defining compounds and/or uses.

The mentioning or published documents does not constitute an admission that these are prior art.

The present invention especially does not relate to a disease which is not ER dependent (meaning that ER activity is at least contributing to the disease, e.g. to the symptoms) in the prophylactic and/or therapeutic treatment.

The following examples illustrate the invention without limiting its scope.

### EXAMPLES

### Example 1

### Preparation of Crude Extracts

1960 g of Smilax myosotiflora roots (SM) were ground into a powder using a lab mill and afterwards extracted at room temperature with 4000 ml 95% Ethanol twice by using ultrasonic. The solution was separated from the remaining material and concentrated under reduced pressure. The remaining water phase was added with water to a final volume of 400 ml and subsequently extracted with n-heptane and Ethyl acetate by liquid/liquid separation.

The n-heptane extract (SM 1 (1) was dried (Na₂SO₄) and the solvent evaporated under reduced pressure. The remaining water phase was extracted with Ethyl acetate for three times. The three Ethyl acetate extracts were combined (SM 1 (2)), dried (Na₂SO₄) and the solvent evaporated under reduced pressure. The remaining water phase (SM 1 (3)) was also evaporated under reduced pressure and the amounts for the three crude extracts were determined:

| Plant | SM-No | Phases | Amount |
|---|---|---|---|
| S. myosotiflora | SM 1 (1) | n-Heptane | 4.8 g |
| S. myosotiflora | SM 1 (2) | Ethyl acetate | 8.0 g |
| S. myosotiflora | SM 1 (3) | Water | 28.2 g |

SM 1 (2) was selected as starting material for isolation of pure compounds.

### Example 2

### Preparation of Pure Compounds

The initial separation steps were performed as MPLC (procedure 3, 9 and 10) separations on reverse phase material (Macherey & Nagel, Dueren, Germany). For the separation of the single compounds in preparative scale a HPLC-setup was used comprising reverse phase separation columns (all provided by Macherey & Nagel, Dueren, Germany). The gradients for elution were chosen according to the separation problem. Generally the systems were based on Water/Acetonitrile mixtures. UV-Signals were detected at 210 nm & 254 nm. Every fraction was dried by using a vacuum concentrator and the yield was determined.

For the control of every single fractionation step the resulting fractions were analysed by HPLC-UV-ELSD.

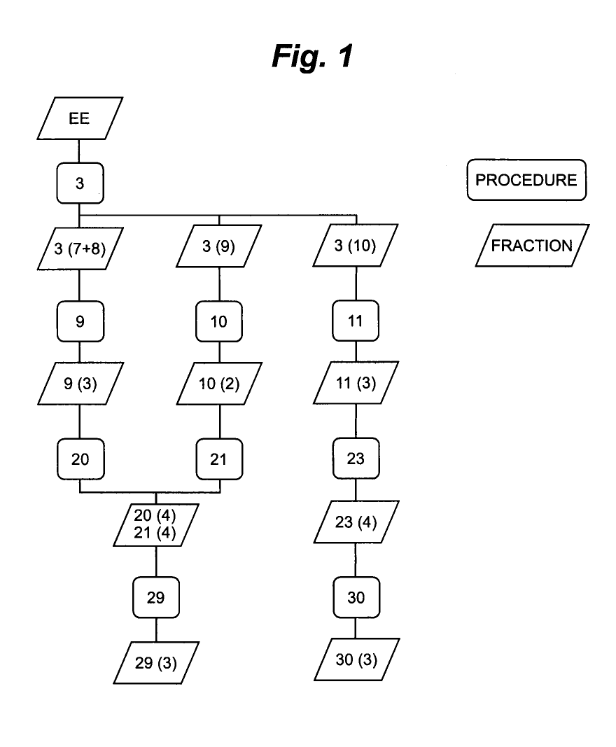

### FIG. 1: Isolation Procedure

TABLE 2
History of isolation

| | | Conditions of separation | Product of | retention time |
|---|---|---|---|---|
| Procedure | Starting | Solvent A: H₂O + 0.1% TFA | separation | period [min], |
| number | Fraction(s) | Solvent B: Acetonitrile + 0.1% TFA | step | yields [mg] |
| 3 | SM 1 (2) | Nucleodur 100-20 C18ec, 130 × 40 mm, | SM 3 (7 + | 51-54 |
| | | Flow: 20 ml/min | 8) | 433 |
| | | Nucleodur 100-20 C18ec, 130 × 40 mm, | SM 3 (9) | 55-57 |
| | | Flow: 20 ml/min | | 228 |
| | | Nucleodur 100-20 C18ec, 130 × 40 mm, | SM 3 (10) | 58-67 |
| | | Flow: 20 ml/min | | 1004 |
| 9 | SM 3 (7 + | Nucleodur 100-20 C18ec, 130 × 40 mm, | SM 9 (3) | 32-67 |
| | 8) | 20 ml/min | | 117 |
| 10 | SM 3 (9) | Nucleosil 100-7 C8ec, 250 × 20 mm, | SM 10 (2) | 5-6 |
| | | 20 ml/min | | 30 |
| 11 | SM 3 (10) | Nucleodur 100-20 C18ec, 130 × 40 mm, | SM 11 (3) | 38-44 |
| | | 20 ml/min | | 202 |
| 20 | SM 9 (3) | Nucleodur 100-5 C18ec, 250 × 20 mm, | SM 20 (4) | 18-19 |
| | | 20 ml/min | | 26 |
| 21 | SM 10 (2) | Nucleosil 100-7 C18, 250 × 10 mm, | SM 21 (4) | 27-29 |
| | | 8 ml/min | | 10 |
| 23 | SM 11 (3) | Nucleodur 100-5 C18ec, 250 × 20 mm, | SM 23 (4) | 17.5-18.5 |
| | | 20 ml/min | | 35 |
| 29 | SM 20 (4) + | Nucleosil 100-7 C18, 250 × 10 mm, | SM 29 (3) | 17.0-17.5 |
| | SM 21 (4) | 8 ml/min | | 7 |
| 30 | SM 23 (4) | Nucleosil 100-7 C18, 250 × 10 mm, | SM 30 (3) | 18.5-19.5 |
| | | 8 ml/min | | 13 |

### Identification Characterisation of the Pure Compounds SM 29 (3) and SM 30 (3):

LC-MS analyses are performed using an Agilent HP1100 (Agilent, Waldbronn, Germany) liquid chromatograph coupled with a LCQ™ Deca XPplus mass spectrometer (Thermo Fisher Scientific, Waltham, Mass., USA) in the positive and negative electrospray ionisation (ESI) mode. A Waters symmetry column is used as stationary phase. Mobile phase A: 0.1% Formic acid in water, mobile phase B: 0.1% Formic acid in acetonitrile; gradient: 0-1 min. 98% A, from 1-21 min. to 100% B, from 21-27 min 100% B. LC-MS spectra are recorded in the range of molecular weights between 160 and 1.600 U.

HR-ESIMS data were obtained on a Bruker MicroTOF instrument, coupled with an HPLC system as described before and using sodium formiate as internal reference.

| | | Molecular | | |
|---|---|---|---|---|
| | Retention | formula | | |
| | time [min] | MW [g/mol] | HRMS [m/z] | Name |
| 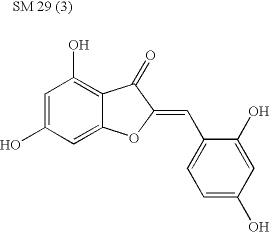 | 10.07 | C₁₅H₁₀O₆ 286.24 | obtained: m/z 287.05587 calculated: m/z 287.0550 [C₁₅H₁₁O₆]⁺, Δ 3.0 ppm | 2′,4,4′,6- Tetrahydroxyaurone |
| 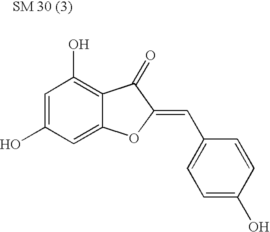 | 10.73 | C₁₅H₁₀O₅ 270.24 | n.d. | 4,4′,6-Trihydroxy- aurone |

### NMR Spectroscopic Data:

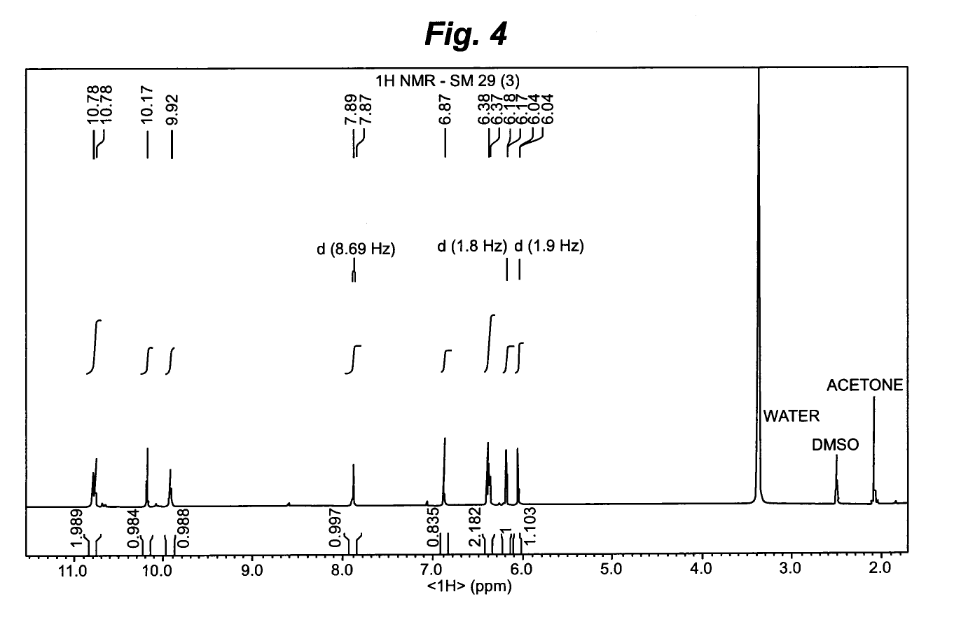

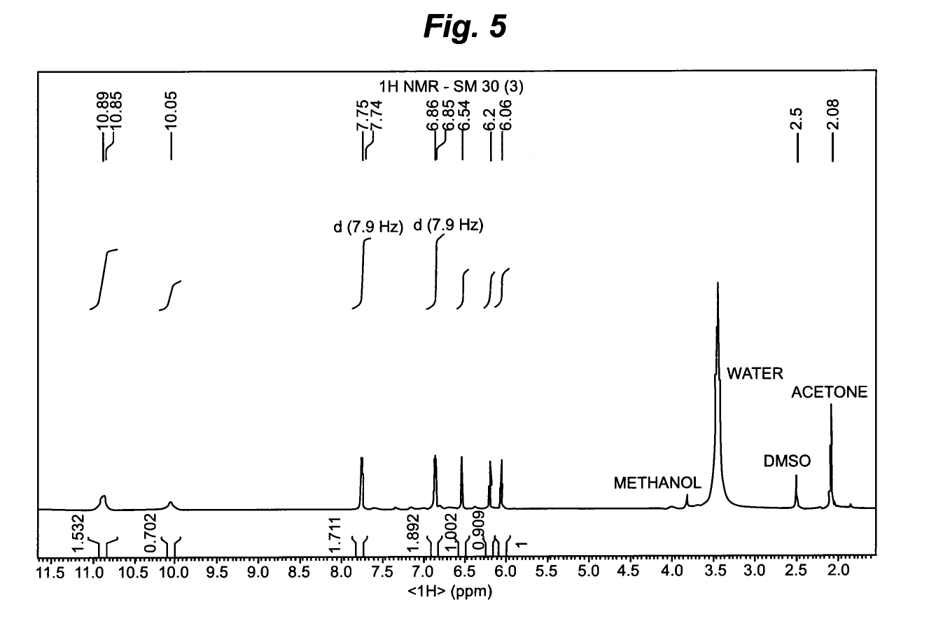

NMR spectra were recorded in DMSO-d₆ on a Bruker DRX500 spectrometer at 293 K, operating at 500.13 MHz proton frequency and 125.76 MHz carbon frequency. The solvent peak was used as internal reference (δ_{H} 2.50, δ_{C} 39.5). Scalar coupling constants J are given in Hertz. ¹H NMR spectra are shown in FIGS. 4 and 5

Structure elucidations and peak assignments are based on thorough analysis of two-dimensional ¹H, ¹H-gCOSY, ¹H, ¹³C-gHSQC, and ¹H¹³C-gHMBC spectra as well as chemical shift interpretation. Furthermore, HPLC-MS data including extracted UV as well as positive and negative mode ESI spectra were used. The molecular formula and elemental composition of novel congener SM 29 (3) was confirmed by high resolution ESIMS experiments (see above). Numbering of the aurone skeleton was done in agreement with the scientific literature (e.g., Jang D S et al., J. Nat. Prod. 2003, 66, 583-587).

| | 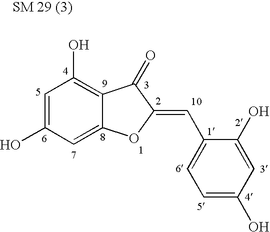 | | 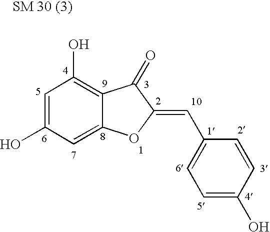 | |
|---|---|---|---|---|
| Atom | ¹³C | ¹H (J in Hz) | ¹³C*⁾ | ¹H (J in Hz) |
| 1 | — | — | — | — |
| 2 | 145.4 | — | 146.5 | — |
| 3 | 179.1 | — | 179.5 | — |
| 4 | 158.0 | — | 157.8 | — |
| 5 | 97.5 | 6.04, d (1.9) | 97.5 | 6.06, d (1.8) |
| 6 | 166.7 | — | 167.1 | — |
| 7 | 90.4 | 6.18, d (1.8) | 90.5 | 6.20, d (1.8) |
| 8 | 167.4 | — | 167.0 | — |
| 9 | 103.0 | — | 103.3 | — |
| 10 | 103.6 | 6.87, s | 109.3 | 6.54, s |
| 1′ | 110.8 | — | 123.8 | — |
| 2′ | 158.6 | — | 132.8 | 7.75, d (7.9) |
| 3′ | 102.3 | 6.39, m | 115.9 | 6.86, d (7.9) |
| 4′ | 160.3 | — | 159.3 | — |
| 5′ | 108.1 | 6.38, m | 115.9 | 6.86, d (7.9) |
| 6′ | 132.0 | 7.88, d (8.7) | 132.8 | 7.75, d (7.9) |
| 2′-OH | — | 10.17, s | — | — |
| 4-OH | — | 10.79, s | — | 10.05, br s |
| 4′-OH | — | 9.92, s | — | 10.89, br s |
| 6-OH | — | 10.76, s | — | 10.86, br s |

*⁾Carbon chemical shifts of SM 30 (3) were obtained from 2-dimentsional HSQC and HMBC experiments.

### Example 3

### Preparation of Enriched Extracts

20 g of Smilax myosotiflora (SM) were ground into a powder using a lab mill (Retsch ZM200, Haan, Germany) and afterwards extracted for 45 min at 40° C. with 50 ml of 75% ethanol in water (v/v) using ultrasonic treatment. Before the water was mixed with the ethanol for the extraction process, the pH value of the water was adjusted to pH 2 by addition of 2M hydrochloric acid. The final pH was checked either with indicator paper (strips: Fisherbrand pH 0-14) and with a pH-meter (WTW pH330).

The extract solution was separated from the remaining material by filtration and the filtrate was concentrated under reduced pressure using a rotary evaporator (max. 40° C. bath temperature; max. 15 mbar; Büchi, Essen, Germany) in order to remove the organic solvent. For enrichment of aurones, the remaining water phase was subjected to further liquid/liquid separation steps.

### First Liquid/Liquid Enrichment Step:

Subsequently the remaining water phase was filled up with water to a final volume of 50 ml and extracted in a first liquid/liquid separation twice with 50 ml ethyl acetate. The two ethyl acetate extract phases were combined (called SM 31(1)), dried (Na_{z} SO₄) and the solvent was evaporated under reduced pressure. The remaining water phase (called SM 31(2)) was also evaporated to dryness. The yields for the dried samples were determined.

### Second Liquid/Liquid Enrichment Step:

In a subsequent second liquid/liquid separation step, further enrichment of the aurones was achieved by re-dissolving 45 mg SM 31(1) in a mixture of 25 ml ethyl acetate and 25 ml PBS (30 mM phosphate buffered saline) at pH 7.4 followed by extraction. The extraction with 25 ml ethyl acetate was repeated. The two ethyl acetate extract phases were combined to yield a product named SM 31(3) and dried (Na_{z} SO₄) and the solvent was evaporated under reduced pressure. The remaining water phase (called SM 31(4)) was also evaporated to dryness. The yields of the dried samples were determined.

### Yields:

| Plant | SM No. | Phase | Amount |
|---|---|---|---|
| S. myosotiflora | SM 31(1) | Ethyl acetate | 49 mg |
| S. myosotiflora | SM 31(2) | Water | 227 mg |
| S. myosotiflora | SM 31(3) | Ethyl acetate | 30 mg |
| S. myosotiflora | SM 31(4) | Water | 15 mg |

Since aurones remain throughout the two liquid/liquid separation processes in the ethyl acetate phases, an enrichment of factor 5.6 in the first step and additional enrichment of factor 1.5 is achieved.

### Example 4

### ERalpha

Recombinant human ERalpha protein isolated from insect 519 cells (MDS Pharma Services: cat No. 226010) was incubated with the test substance (dissolved in 1% DMSO) and 0.5 nM [³H] estradiol for 2 hours at 25° C. in an incubation buffer (10 mM TrisHCl pH 7.4, 0.1% BSA, 10% Glycerol, 1 mM DTT). At the end of the incubation period, receptor-bound [³H] estradiol was quantified. Stimulating or inhibiting effects equal or larger than 50% in comparison to vehicle control (1% DMSO) were considered to be significant. The final concentration of the test compound was 10 μM.

For compound SM30 (3), 18% inhibition of [³H] estradiol binding to ERalpha was determined (in comparison to the vehicle control); for SM29 (3), 2% inhibition of binding was measured.

### Example 5

### ERbeta

Recombinant human ERbeta protein isolated from insect Sf9 cells (MDS Pharma Services: cat No. 226050) was incubated with the test substance (dissolved in 1% DMSO) and 0.5 nM [³H] estradiol for 2 hours at 25° C. in an incubation buffer (10 mM TrisHCl pH 7.4, 0.1% BSA, 10% Glycerol, 1 mM DTT). At the end of the incubation period, receptor-bound [³H] estradiol was quantified. Stimulating or inhibiting effects equal or larger than 50% in comparison to vehicle control (1% DMSO) were considered to be significant. The final concentration of the test compound was 10 μM.

For compound SM30 (3), 91% inhibition of [³H] estradiol binding to ERbeta was determined (in comparison to the vehicle control); for SM29 (3), 13% inhibition of binding was measured.

The assays described in Examples 4 and 5 were conducted by MDS (catalogue number 226010 for ERalpha and 226050 for ERbeta, respectively) following procedures established in literature (Obourn et al., Biochemistry 1993, 32: 6229-6236).

1. A compound of the formula I, 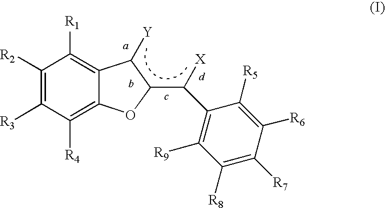
wherein each of R₁ to R₉ is, independently of the others, H, hydroxy, fluoro, chloro, bromo, iodo, C₁-C₈-alkyl, C₂-C₈-alkenyl, C₂-C₈-alkynyl, C₃-C₁₀-cycloalkyl, phenyloxy, C₁-C₈-alkoxy, C₁-C₉-alkanoyloxy, benzoyl or the radical of a C₅-C₁₂-carbohydrate bound via one of its oxygen atoms, where alkyl, alkenyl, alkynyl, cycloalkyl, phenyl, alkoxy, alkanoyloxy and benzoyl can be unsubstituted or substituted by one, two or three substituents selected independently of each other from the group consisting of —F, —Cl, —Br, —I, —OH, —OCH₃, OCH₂CH₃, OCOCH₃, —CH₃, —CHO, and CO₂H, or the radical of a C₅-C₁₂-carbohydrate bound via one of its oxygen atoms, preferably with the proviso that if R₁, R₃ and R₇ each are bound via an oxygen, R₂, R₄, R₅ and R₉ each are hydrogen and one of R₆ and R₈ is bound via an oxygen, then the other of R₆ and R₈ has one of the meanings mentioned above other than H;
where one of R₁ to R₉ may, in addition, be a substitutent of the subformula IA 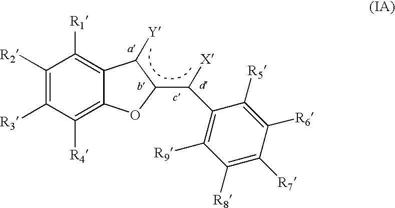
wherein one of R₁′ to R₉′ forms the bond to the rest of the molecule in formula I, while the others are, independently of each other, H, hydroxy, fluoro, chloro, bromo, iodo, C₁-C₈-alkyl, C₂-C₈-alkenyl, C₂-C₈-alkynyl, C₃-C₁₀-cycloalkyl, phenyloxy, C₁-C₈-alkoxy, C₁-C₉-alkanoyloxy, benzoyl or the radical of a C₅-C₁₂-carbohydrate bound via one of its oxygen atoms, where alkyl, alkenyl, alkynyl, cycloalkyl, phenyl, alkoxy, alkanoyloxy and benzoyl can be unsubstituted or substituted by one, two or three substituents selected independently of each other from the group consisting of —F, —Cl, —Br, —I, —OH, —OCH₃, —OCH₂CH₃, —OCOCH₃, —CHO, and —CO₂H;
or two adjacent moieties of R₁ to R₉ and of R₁′ to R₉′ together form a —O—CH₂—O— or a —O—CH₂—CH₂—O— bridge, thus forming with the two atoms to which they are bound a ring, while the other moieties are independently selected from those mentioned above;
in formula I either bond a and bond c each are a double bond, or bonds b and bond d each are a double bond, respectively;
and, if present, in subformula IA either bond a′ and bond c′ each are a double bond, or bonds b′ and bond d′ each are a double bond, respectively;
where the double bonds in formula I and, if present, subformula IA, may also be in tautomeric equilibrium;
X is hydrogen, oxo, hydroxy, C₁-C₈-alkoxy, especially methoxy, C₁-C₈-alkanoyloxy, especially acetyloxy, benzoyloxy or 3,4,5-trihydroxybenzoyloxy, or, if bonds a and c are double bonds in formula I and Y is oxo, can also be a moiety of the subformula IB, 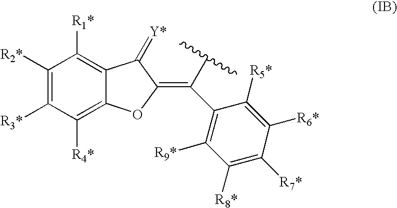
wherein the waved line indicates the end of the bond where said moiety of the subformula IB is bound to the rest of the molecule of formula I and wherein
Y* is oxo and
R₁* to R₉* are, independently of each other, H, hydroxy, fluoro, chloro, bromo, iodo, C₁-C₈-alkyl, phenyloxy, C₁-C₈-alkoxy, C₁-C₉-alkanoyloxy, benzoyl or the radical of a C₅-C₁₂-carbohydrate bound via one of its oxygen atoms;
and Y is oxo, hydroxy or C₁-C₈-alkoxy;
a mixture of two or more compounds of the formula I, and/or an extract comprising one or more compounds of the formula I, for use in the prophylactic and/or therapeutic treatment of an animal with a estrogen receptor (ER) related disease or condition;
where the compounds of the formula I may be present in free form, in the form of a pharmaceutically and/or nutraceutically acceptable salt, in the form of a tautomer, in the form of an ester and/or in the form of a solvate.

2. A compound for use of the formula I, mixture of compounds of the formula I or extract of the formula I use according to claim 1, wherein
Y is oxo,
X is H, hydroxy, methoxy, acetoxy, benzoyloxy or 3,4,5-trihydroxybenzoyloxy,
the bonds a and c are double bonds, respectively, the bonds b and d are single bonds, respectively, where the compounds of the formula I may be present in free form, in the form of a pharmaceutically and/or nutraceutically acceptable salt, in the form of a tautomer, in the form of an ester and/or in the form of a solvate.

3. A compound for use of the formula I, mixture of compounds of the formula I or extract of the formula I according to claim 1, wherein
each of R₁ to R₉ is, independently of the others, H, hydroxy, chloro, C₁-C₈-alkyl, C₁-C₈-alkoxy, C₁-C₉-alkanoyloxy or benzoyl with the proviso that if R₁, R₃ and R₇ each are hydroxyl, R₂, R₄, R₅ and R₉ each are hydrogen and one of R₆ and R₈ is hydroxy, then the other of R₆ and R₈ has one of the meanings mentioned above other than H;
Y is oxo, and
X is H, hydroxy, methoxy, acetoxy, benzoyloxy or 3,4,5-trihydroxybenzoyloxy,
the bonds a and c are double bonds, respectively, the bonds b and d are single bonds,
where the compounds of the formula I may be present in free form, in the form of a pharmaceutically and/or nutraceutically acceptable salt, in the form of a tautomer, in the form of an ester and/or in the form of a solvate.

4. A compound of the formula I, mixture of compounds of the formula I and/or extract of the formula I for use according to claim 1, where the compound(s) of the formula I is or are selected from the group consisting of those with the following formulae: 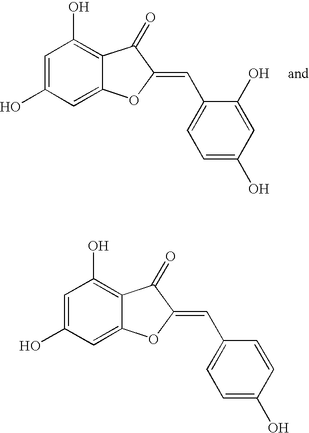
where the compound(s) may be present in free form, in the form of a tautomer, in the form of an ester and/or in the form of a solvate.

5. An extract comprising one or more compounds of the formula I in free or any other form mentioned for use according to claim 1 which is obtained from Smilax myosotiflora, especially from the roots.

6. An extraction and purification or at least enrichment process for obtaining a compounds or an extract according to claim 1 comprising
an extraction step from a plant or plant parts, where the plant is of the Genus Smilax, and
a first liquid/liquid separation step under acidic conditions, followed by
a subsequent second liquid/liquid extraction of the material found in the less polar phase of the first extraction step, preferably under neutral to slightly alkaline conditions, where preferably
said extraction and said first liquid/liquid separation step takes place under acidic conditions at a pH of about 2 to about 4.5, more preferably about 2 to about 3, most preferably at a pH of about 2a followed by said second liquid/liquid extraction step performed at a pH of about 7 or larger.

7. The extraction and purification or at least enrichment process for obtaining a compounds or an extract according to claim 6, wherein said first liquid/liquid separation step takes place at a pH of about 2 to about 3.

8. The extraction and purification or at least enrichment process for obtaining a compounds or an extract according to claim 6, wherein said first liquid/liquid separation step takes place at a pH of about 2.

9. The extraction and purification or at least enrichment process for obtaining a compounds or an extract according to claim 6, wherein said second liquid/liquid separation step takes place at a pH of about 7 to about 9.

10. The extraction and purification or at least enrichment process for obtaining a compounds or an extract according to claim 6, wherein said second liquid/liquid separation step takes place at a pH of about 7.4 to about 7.6.

11. A compound of the formula I, mixture of compounds of the formula I and/or extract of the formula I for use according to claim 1, wherein the compound or compounds according to formula I are present in an amount of 10 or more % by weight.

12. A compound of the formula I, mixture of compounds of the formula I and/or extract of the formula I for use according to claim 1, wherein the compound or compounds according to formula I are present in an amount of 30 or more % by weight.

13. A compound of the formula I, mixture of compounds of the formula I and/or extract of the formula I for use according to claim 1, wherein the compound or compounds according to formula I are present in an amount of 50% or more by weight.

14. A compound of the formula I, mixture of compounds of the formula I and/or extract of the formula I for use according to claim 1, wherein the compound or compounds according to formula I are present in an amount of 80 to 100% by weight.

15. A compound or extract for use according to claim 1 where the compound or extract shows at a concentration of 10 μM an at least 3-fold stronger inhibition of the binding of estrogen to ERbeta than to ERalpha.

16. A pharmaceutical composition or a nutraceutical comprising a compound of the formula I, a mixture of compounds of the formula I or an extract of the formula I according to claim 1 for use in the prophylactic and/or therapeutic treatment of an animal with a estrogen receptor (ER) related disease or condition, together with at least one pharmaceutically or nutraceutically acceptable carrier material.

17. The composition according to claim 16, where the compound(s) of formula I are present in an amount of 0.001 to 100% by weight.

18. The composition according to claim 16, where the compound(s) of formula I are present in an amount. from 0.1 to 50% by weight.

19. A method of prophylactically and/or therapeutically treating an animal, especially a human in need of such treatment, of using a compound of the formula I, presumed to suffer in future or suffering from a estrogen receptor (ER) related disease or condition, comprising administering to said animal or human an effective amount of a compound of the formula I, a compound mixture or an extract according to claim 1 in free or other forms mentioned therein.

20. The use of a compound of the formula I, or a mixture of compounds of the formula I, or an extract comprising one or more compounds of the formula I, as defined in claim 1 in free or other forms mentioned therein, as active ingredient for the manufacture of a medicament or nutraceutical or food supplement for the treatment of a ER related disease or condition.

21. The use of a compound of the formula I, or a mixture of compounds of the formula I, or an extract comprising one or more compounds of the formula I, as defined in claim 1 in free or other forms mentioned therein, as active ingredient in the treatment of a ER related disease or condition.

22. The compound, compound mixture or extract in free or any other form mentioned according to claim 1, where the estrogen receptor related disease or disorder is selected from the group consisting of bone loss, bone fractures, osteoporosis, Paget's disease, periodontal disease, hot flashes, cardiovascular disease, restenosis, vascular smooth muscle cell proliferation, obesity, incontinence, multiple sclerosis, sexual dysfunction, hypertension and retinal degeneration.

23. The compound, compound mixture or extract in free or any other form mentioned according to claim 1, where the estrogen receptor related disease or disorder is selected from the group consisting of impairment of cognitive functioning, age-related mild cognitive impairment, cerebral degenerative disorders, anxiety, depression, perimenopausal depression, post-partum depression, premenstrual syndrome, manic depression, anxiety, dementia, obsessive compulsive behavior, attention deficit disorder, sleep disorders, irritability, impulsivity and anger management.
